(19) United States
(12) Patent Application Publication
(10) Pub. No.: US 2023/0410984 A1
(43) Pub. Date: Dec. 21, 2023

(54) METHODS AND SYSTEMS FOR IMAGING

(71) Applicant: SHANGHAI UNITED IMAGING HEALTHCARE CO., LTD., Shanghai (CN)

(72) Inventors: Qiujing XIANG, Shanghai (CN); Guobin LI, Shanghai (CN)

(73) Assignee: SHANGHAI UNITED IMAGING HEALTHCARE CO., LTD., Shanghai (CN)

(21) Appl. No.: 18/338,337

(22) Filed: Jun. 20, 2023

(30) Foreign Application Priority Data
Jun. 20, 2022 (CN) 202210696865.0
Jun. 23, 2022 (CN) 202210728055.9
Jun. 23, 2022 (CN) 202210737924.4

(51) Int. Cl. G16H 30/20; G16H 40/40; G06V 10/75

(52) U.S. Cl. CPC G16H 30/20; G16H 40/40; G06V 10/759; G06V 40/172

(57) ABSTRACT

Embodiments of the present disclosure provide a method, a system and a non-transitory computer readable medium for imaging. The method may include: obtaining historical configuration information; adjusting the historical configuration information to generate target configuration information matching a current scanning condition; and performing, using a first device, an imaging operation on the detection object based on the target configuration information, wherein an account, a current scanning device, an operator, or a detection object under the current scanning condition is different from an account, the historical scanning device, an operator, or a detection object under the historical scanning condition, respectively.

### CROSS-REFERENCE TO RELATED APPLICATIONS

This application claims priority to Chinese Patent Application No. 202210696865.0, filed on Jun. 20, 2022, Chinese Patent Application No. 202210728055.9, filed on Jun. 23, 2022, and Chinese Patent Application No. 202210737924.4, filed on Jun. 23, 2022, the entire contents of each of which are incorporated herein by reference.

### TECHNICAL FIELD

The present disclosure relates to the field of medical technology, in particular, to methods and systems for imaging.

### BACKGROUND

In the process of medical imaging, to provide a better user experience, users can freely configure the options on the device based on their own work habits and preferences, greatly enhancing the user's experience with the device. However, in many cases, there are multiple devices within an institution, and when a user sets the configuration options on one device, they need to set them again on another device, leading to a large workload for the user and low data synchronization efficiency.

Furthermore, the consistency of displaying and presenting medical images ensures consistent interpretation and diagnosis of patient images, and ensures the normal and smooth progress of patient diagnosis and treatment. However, when the same patient seeks medical care in different hospitals or at different times, examinations may be conducted on different scanning devices. Due to differences in equipment purchased from different manufacturers by different medical institutions and variations in the proficiency of equipment operators, medical images obtained on different scanning devices may exhibit varying degrees of differences. These differences can affect the judgment of clinical doctors and reduce the accuracy of patient re-examinations.

Therefore, it is desirable to develop an imaging solution that can improve the accuracy and efficiency of imaging for different users on different devices when imaging different objects.

### SUMMARY

An aspect of the present disclosure provides a method implemented on at least one machine each of which has at least one processor and at least one storage device for imaging. The method may include obtaining historical configuration information, the historical configuration information including at least one of a historical inspection process configuration, a historical scanning scheme configuration, a historical displaying configuration, or twin data of a historical detection object used when a historical scanning device is running in a historical scanning condition; adjusting the historical configuration information to generate target configuration information matching a current scanning condition, the target configuration information including at least one of an inspection process configuration, a scanning scheme configuration, a displaying configuration, or twin data of a detection object; and performing, using a first device, an imaging operation on the detection object based on the target configuration information, wherein an account, a current scanning device, an operator, or a detection object under the current scanning condition may be different from an account, the historical scanning device, an operator, or a detection object under the historical scanning condition, respectively.

Another aspect of the present disclosure provides a system for imaging. The system may include an acquisition module configured to obtain historical configuration information, and obtain target configuration information; and an imaging module configured to perform an imaging operation using a first device based on the target configuration information, wherein the target configuration information includes at least one of inspection process configuration, scanning scheme configuration, displaying configuration, and twin data of a detection object.

Another aspect of the present disclosure provides a non-transitory computer readable medium storing instructions, the instructions, when executed by at least one processor, causing the at least one processor to implement a method including: obtaining historical configuration information, the historical configuration information including at least one of a historical inspection process configuration, a historical scanning scheme configuration, a historical displaying configuration, or twin data of a historical detection object used when a historical scanning device is running in a historical scanning condition; adjusting the historical configuration information to generate target configuration information matching a current scanning condition, the target configuration information including at least one of an inspection process configuration, a scanning scheme configuration, a displaying configuration, or twin data of a detection object; and performing, using a first device, an imaging operation on the detection object based on the target configuration information, wherein an account, a current scanning device, an operator, or a detection object under the current scanning condition may be different from an account, the historical scanning device, an operator, or a detection object under the historical scanning condition, respectively.

Another aspect of the present disclosure provides a method implemented on a first device for data migration. The method may include obtaining a target account via a login interface of the first device; obtaining a target preference setting corresponding to the target account, the target preference setting including a setting of one or more configuration options on a second device, the setting of the one or more configuration options being set by an operator on the second device after the operator starts the second device using the target account, the one or more configuration options including at least one of an inspection process configuration, a scanning scheme configuration, or a displaying configuration; updating a setting corresponding to at least one configuration option on the first device based on the target preference setting.

Another aspect of the present disclosure provides a method implemented on a server for data migration. The method may include receiving a first acquisition request sent by a first device, wherein the first acquisition request carries a target account, the target account being obtained via a login interface of the first device; searching, based on the target account and a first mapping relationship, for a preference setting corresponding to the target account, the first mapping relationship indicating a correlation between a plurality of accounts and a plurality of preference settings; designating the preference setting as a target preference setting corresponding to the target account, wherein the target preference setting may include a setting of one or more configuration options on a second device, the setting of the one or more configuration options being set by an operator on the second device using the target account, and the one or more configuration options including at least one of an inspection process configuration, a scanning scheme configuration, or a displaying configuration; sending the target preference setting to the first device to facilitate the first device to update a setting corresponding to at least one configuration option on the first device based on the target preference setting; and starting the first device.

Another aspect of the present disclosure provides a method implemented on a second device for data migration. The method may include receiving a second acquisition request sent by a first device, wherein the second acquisition request carries a target account, and the second acquisition request is sent by the first device based on a device identification of the second device; searching, based on the target account and a second mapping relationship, for a preference setting corresponding to the target account, the second mapping relationship indicating correlations between a plurality of accounts and a plurality of preference settings; designating the preference setting as a target preference setting corresponding to the target account, wherein the target preference setting includes a setting of one or more configuration options on a second device, the setting of the one or more configuration options being set by an operator on the second device using the target account, and the one or more configuration options including at least one of an inspection process configuration, a scanning scheme configuration, or a displaying configuration; sending the target preference setting to the first device to facilitate the first device to update a setting corresponding to at least one configuration option on the first device based on the target preference setting; and starting the first device.

Another aspect of the present disclosure provides a terminal. The terminal may include a receiving module configured to receive a second configuration acquisition request sent by a first device, wherein the second configuration acquisition request carries a target account, and the second configuration acquisition request is sent by the first device based on a device identification of a second device; a searching module configured to search for a preference setting corresponding to a target account in a second mapping relationship based on the target account, and designate the preference setting as the target preference settings, wherein the second mapping relationship is used to indicate a correlation between an account and a preference setting, and the target preference setting is a result of setting a configuration option on a second device after a user starts the second device using the target account, and the configuration option include at least one of inspection process configuration, scanning scheme configuration, and displaying configuration; a sending module configured to send the target preference setting to the first device to drive the first device to update a setting corresponding to a configuration option on the first device based on the target preference setting, and start the first device.

Another aspect of the present disclosure provides a method for adjusting an image scanning scheme. The method may include obtaining twin data of a detection object, the twin data of the detection object including portrait data of the detection object; determining, based on the twin data of the detection object, an initial image scanning scheme corresponding to the twin data of the detection object, the initial image scanning scheme including a plurality of scanning phases; executing the initial image scanning scheme; determining whether the portrait data of the detection object has changed during a current scanning phase of the plurality of scanning phases; in response to a determination that the portrait data of the detection object has changed, optimizing one or more parameters of at least one pending scanning phase of the plurality of scanning phases in the initial image scanning scheme.

Another aspect of the present disclosure provides a non-transitory computer readable medium storing instructions, the instructions, when executed by at least one processor, causing the at least one processor to implement a method including: obtaining twin data of a detection object, the twin data of the detection object including portrait data of the detection object; determining, based on the twin data of the detection object, an initial image scanning scheme corresponding to the twin data of the detection object, the initial image scanning scheme including a plurality of scanning phases; executing the initial image scanning scheme; determining whether the portrait data of the detection object has changed during a current scanning phase of the plurality of scanning phases; in response to a determination that the portrait data of the detection object has changed, optimizing one or more parameters of at least one pending scanning phase of the plurality of scanning phases in the initial image scanning scheme.

Another aspect of the present disclosure provides a method for generating an image scanning scheme. The method may include obtaining historical scanning scheme data, the historical scanning scheme data including twin data of a historical object and twin data of a historical operator; obtaining portrait data of a current object and initial twin data of a current operator; fusing the historical scanning scheme data with the portrait data of the current object and the initial twin data of the current operator to generate an image scanning scheme for the current object.

Another aspect of the present disclosure provides a non-transitory computer readable medium storing instructions, the instructions, when executed by at least one processor, causing the at least one processor to implement a method including: obtaining historical scanning scheme data, the historical scanning scheme data including twin data of a historical object and twin data of a historical operator; obtaining portrait data of a current object and initial twin data of a current operator; fusing the historical scanning scheme data with the portrait data of the current object and the initial twin data of the current operator to generate an image scanning scheme for the current object.

### BRIEF DESCRIPTION OF THE DRAWINGS

The present disclosure is further illustrated in terms of exemplary embodiments, and these exemplary embodiments are described in detail with reference to the drawings. These embodiments are not restrictive. In these embodiments, the same number indicates the same structure, wherein:

FIG. 1 is a schematic diagram illustrating an exemplary application scenario of a system for imaging according to some embodiments of the present disclosure;

FIG. 2 is a block diagram illustrating an exemplary system for imaging according to some embodiments of the present disclosure;

FIG. 3 is a flowchart an exemplary process of imaging according to some embodiments of the present disclosure;

FIG. 4 is a flowchart illustrating an exemplary process for data migration implemented on a first device according to some embodiments of the present disclosure;

FIG. 5 is a flowchart illustrating an exemplary process for data migration implemented on a server according to some embodiments of the present disclosure;

FIG. 6 is a flowchart illustrating an exemplary process for data migration implemented on a second device according to some embodiments of the present disclosure;

FIG. 7 is a block diagram illustrating an exemplary terminal for data migration according to some embodiments of the present disclosure;

FIG. 8 is a flowchart illustrating an exemplary process for updating setting(s) of configuration option(s) on a first device according to some embodiments of the present disclosure;

FIG. 9 is a schematic diagram illustrating an exemplary login interface of a first device according to some embodiments of the present disclosure;

FIG. 10 is a schematic diagram illustrating an exemplary interface for inputting a device identification of a first device according to some embodiments of the present disclosure;

FIG. 11 is a schematic diagram illustrating an exemplary second mapping relationship according to some embodiments of the present disclosure;

FIG. 12 is a schematic diagram illustrating an exemplary first mapping relationship according to some embodiments of the present disclosure;

FIG. 13 is a flowchart illustrating an exemplary process for optimizing an image scanning scheme based on twin data of a detection object according to some embodiments of the present disclosure;

FIG. 14 is a flowchart illustrating an exemplary process for generating an image scanning scheme based on historical scanning schema data according to some embodiments of the present disclosure;

FIG. 15 is a flowchart illustrating an exemplary process for generating an image scanning scheme for a current object when the current object is the same as a historical object, and a scanning region of interest (ROI) of the current object is the same as a scanning ROI of the historical object;

FIG. 16 is a flowchart illustrating an exemplary process for generating an image scanning scheme for a current object when the current object is the same as a historical object, and a scanning ROI of the current object is different from a scanning ROI of the historical object; and

FIG. 17 is a flowchart illustrating an exemplary process for generating an image scanning scheme for a current object when the current object is different from a historical object, and a scanning ROI of the current object is the same as a scanning ROI of the historical object.

### DETAILED DESCRIPTION

In order to illustrate the technical solutions related to the embodiments of the present disclosure, a brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless stated otherwise or obvious from the context, the same reference numeral in the drawings refers to the same structure and operation.

It will be understood that the terms “system,” “device,” “unit,” and/or “module” used herein are one method to distinguish different components, elements, parts, sections, or assemblies of different levels in ascending order. However, the terms may be displaced by other expressions if they may achieve the same purpose.

As shown in the present disclosure and claims, unless the context clearly indicates exceptions, the words “a,” “an,” “one,” and/or “the” do not specifically refer to the singular, but may also include the plural. The terms “including” and “comprising” only suggest that the steps and elements that have been clearly identified are included, and these steps and elements do not constitute an exclusive list, and the method or device may also include other steps or elements.

The flowcharts used in the present disclosure may illustrate operations executed by the system according to embodiments in the present disclosure. It should be understood that a previous operation or a subsequent operation of the flowcharts may not be accurately implemented in order. Conversely, various operations may be performed in inverted order, or simultaneously. Moreover, other operations may be added to the flowcharts, and one or more operations may be removed from the flowcharts.

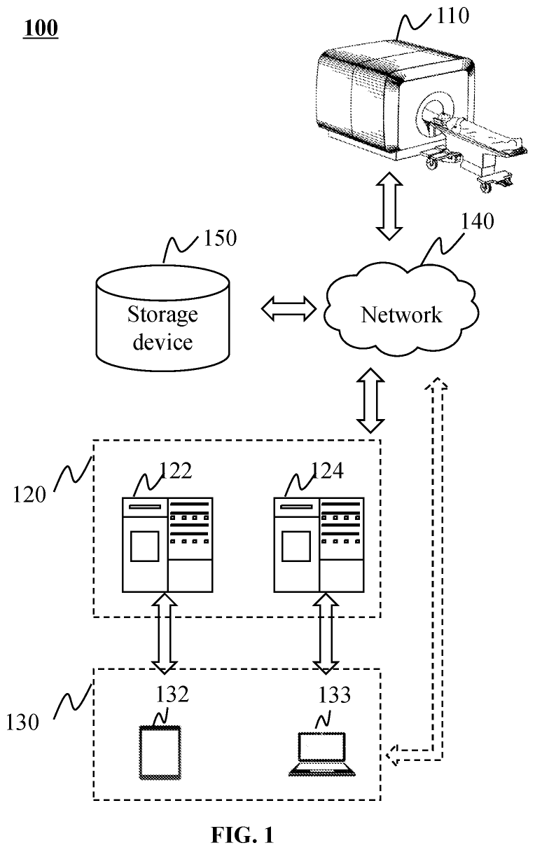

FIG. 1 is a schematic diagram illustrating an exemplary application scenario 100 of a system for imaging according to some embodiments of the present disclosure.

As shown in FIG. 1, the application scenario of the system 100 for imaging may include a scanning device 110, a processing device 120, a terminal 130, a network 140, and a storage device 150. In some embodiments, the system 100 may be used for imaging.

The scanning device 110 may transmit signal(s) (e.g., radiation ray(s)) to a target object or a part thereof and collect data related to the target object or a part thereof. In some embodiments, the target object may include, but may not be limited to a human body, an organ, a body, an injured part, a tumor, an object, a phantom, etc. In some embodiments, the scanning device 110 may include a single mode scanning device. For example, the scanning device 110 may include a magnetic resonance imaging (MRI) device (also known as a MR scanner), a digital subtraction angiography (DSA), a positron emission tomography (PET) device, a single photon emission computed tomography (SPECT) device, a computed tomography (CT) device, an ultrasound scanner, a digital radiography (DR) scanner, or any combination thereof. In some embodiments, the scanning device 110 may include a multimodal scanning device. For example, the multimodal scanning device may include a PET-CT device, a PET-MR device, or any combination thereof. In some embodiments, the scanning device 110 may be arranged in a scanning room.

The processing device 120 may be a device that obtains scanned images based on scanned data. In some embodiments, the processing device 120 may be arranged in an operation room. In some embodiments, the processing device 120 may include a first device 112 and/or a second device 114. The first device 112 may be a device that a user (also referred to as an operator (e.g., a technician)) is currently using. The second device 114 may be a device that the user has previously used. In some embodiments, the user may have completed settings of configuration options on the second device 114.

In some embodiments, the processing device 120 may obtain data and/or information from the scanning device 110, the terminal 130, and/or the storage device 150. For example, the processing device 110 may obtain scanning data from the scanning device 110. For example, the first device 112 may obtain a target preference setting from the second device 114. For example, the first device 112 may obtain historical scanning scheme data from the storage device 150.

In some embodiments, the processing device 120 may include a central processing unit (CPU), a graphics processing unit (GPU), a digital signal processor (DSP), a system on chip (SoC), a microcontroller unit (MCU), and/or any combination thereof. In some embodiments, the processing device 120 may include a computer, a user console, a single server or a server group. The server group may be centralized or distributed. In some embodiments, the processing device 120 may be local or remote. For example, the processing device 120 may access information and/or data stored in the scanning device 110, the terminal 130, and/or the storage device 130 via the network 140. As another example, the processing device 120 may access the information and/or data by directly connecting to the scanning device 110, the terminal 130, and/or the storage device 130. In some embodiments, the processing device 120 may be implemented on a cloud platform. Merely by way of example, the cloud platform may include a private cloud, a public cloud, a hybrid cloud, a community cloud, a distributed cloud, an internal cloud, a multi-layer cloud, or the like, or any combination thereof. In some embodiments, the processing device 120 or a portion of the processing device 120 may be integrated into the scanning device 110.

The terminal 130 may display data (e.g., image(s), pop-up window(s), and prompt information) to the user and/or receive data (e.g., device identification) input by the user. In some embodiments, the terminal 130 may include a first mobile terminal 132 and/or a second mobile terminal 134. The first mobile terminal 132 may be connected with or in communication with the first device, and the second mobile terminal 134 may be connected with or in communication with the second device. The terminal 130 may include a mobile device 131, a tablet computer 132, a laptop computer 133, or any combination thereof. In some embodiments, the terminal 130 may be a part of the processing device 120.

The network 140 may include any suitable network that may facilitate the exchange of the information and/or data for the system for imaging. In some embodiments, one or more components (e.g., the scanning device 110, the processing device 120, the terminal 130, and the storage device 150) of the system for imaging may exchange information and/or data with one or more other components of the system for imaging via the network 140. For example, the processing device 120 may obtain the scanning data from the scanning device 110 via the network 140. In some embodiments, the one or more components of a imaging system (e.g., the scanning device 110, the processing device 120, the terminal 130, and the storage device 150) may communicate information and/or data with one or more external resources (such as third-party external databases, etc.). The network 140 may be and/or include a public network, a private network, a wired network, a wireless network, a cellular network, a frame relay network, a virtual private network (“VPN”), a satellite network, a telephone network, a router, a hub, a switch, a server computer, or any combination thereof. In some embodiments, the network 140 may include one or more network access points. For example, the network 140 may include a base station and/or an Internet exchange point, through which one or more components of the system for imaging may be connected to the network 140 to exchange the data or the information.

The storage device 150 may store data, instructions, and/or any other information. In some embodiments, the storage device 150 may include one or more databases. For example, a database may include a backend database operated by an operator. For example, a database may also include an object information database of a hospital. In some embodiments, the storage device 150 may store data obtained from the scanning device 110, terminal 130, and/or processing device 120. In some embodiments, the storage device 150 may store data and/or instructions, and processing device 120 may execute or use the data and instructions to execute the exemplary method/system described in the present disclosure. In some embodiments, the storage device 150 may include a large capacity memory, a removable memory, a volatile read write memory, a read-only memory (ROM), or any combination thereof. In some embodiments, the storage device 150 may be connected to the network 140 to communicate with one or more other components of the system for imaging (e.g., the scanning device 110, the processing device 120, the terminal 130, and the storage device 150). The one or more components of the system for imaging may access data or instructions stored in storage device 150 via the network 140. In some embodiments, the storage device 150 may be directly connected to or communicate with one or more other components of the imaging system (e.g., the scanning device 110, the processing device 120, the terminal 130, and the storage device 150). In some embodiments, the storage device 150 may be a part of the processing device 120.

It should be noted that the above descriptions are merely provided for illustrative purposes and may not be intended to limit the scope of the present disclosure. For those of ordinary skill in this field, various changes and modifications may be made under the guidance of the present disclosure. The features, structures, methods, and other features of the exemplary embodiments described herein may be combined in various ways to obtain additional or alternative exemplary embodiments to obtain additional and/or alternative exemplary embodiments. However, these changes and modifications may not deviate from the scope of the present disclosure.

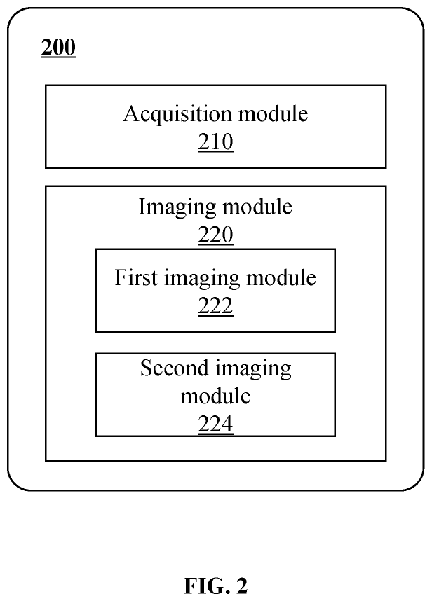

FIG. 2 is a block diagram illustrating an exemplary system 200 for imaging according to some embodiments of the present disclosure. As show in FIG. 2, the system 200 may include an acquisition module 210 and an imaging module 220.

In some embodiments, the acquisition module 210 may be configured to obtain historical configuration information. In some embodiments, the historical configuration information may include at least one of a historical inspection process configuration, a historical scanning scheme configuration, a historical displaying configuration, and twin data of a historical detection object used when a historical scanning device is running in a historical scanning condition. In some embodiment, the acquisition module 210 may be configured to adjust the historical configuration information to generate target configuration information matching a current scanning condition. Corresponding to the historical configuration information, the target configuration information may include at least one of an inspection process configuration, a scanning scheme configuration, a displaying configuration, or twin data of a detection object. In some embodiments, the an account, a current scanning device, an operator, or a detection object under the current scanning condition is different from an account, the historical scanning device, an operator, or a detection object under the historical scanning condition, respectively. In some embodiments, the acquisition module 210 may obtain a target account, the target account matching with the target configuration information, and update a setting corresponding to at least one configuration option on the first device based on a target preference setting related to the inspection process configuration, the scanning scheme configuration, and/or the displaying configuration in the target configuration information. In some embodiments, the target preference setting may include a setting of one or more configuration options on a second device, the setting of the one or more configuration options being set by an operator on the second device using a target account. In some embodiments, the acquisition module 210 may package the target configuration information in a second device; upload the packaged target configuration information to a cloud server via the second device or a second mobile terminal, the second mobile terminal being in communication with or connected with the second device; and download the packaged target configuration information from the cloud server via the first device or a first mobile terminal to the first device, the first mobile terminal being in communication with or connected with the first device. In some embodiments, the acquisition module 210 may package the target configuration information in a second device; store the packaged target configuration information in a storage medium or a third mobile terminal; and obtain the packaged target configuration information from the storage medium or the third mobile terminal via the first device.

In some embodiments, the acquisition module 210 may obtain a target account via a login interface of the first device; obtain a target preference setting corresponding to the target account, the target preference setting including a setting of one or more configuration options on a second device, the setting of the one or more configuration options being set by an operator on the second device after the operator starts the second device using the target account, the one or more configuration options including at least one of an inspection process configuration, a scanning scheme configuration, or a displaying configuration; and update a setting corresponding to at least one configuration option on the first device based on the target preference setting.

In some embodiments, the acquisition module 210 may send a first acquisition request to a server, wherein the first acquisition request carries the target account to facilitate the server to search, based on the target account and a first mapping relationship, for the target preference setting corresponding to the target account, the first mapping relationship indicating a correlation between a plurality of accounts and a plurality of preference settings; and receive the target preference setting sent by the server. In some embodiments, the acquisition module 210 may obtain a device identification of the second device; determine, based on the device identification of the second device, whether the target preference setting is compatible with the first device; and in response to a determination that the target preference setting is incompatible with the first device, adjust the target preference setting; and update the setting corresponding to the at least one configuration option on the first device based on the adjusted target preference setting. In some embodiments, the acquisition module 210 may receive the target account input by the operator on the login interface of the first device; and in response to a login operation triggered by the operator on the login interface of the first device, pop up a data migration pop-up window, the data migration pop-up window including a confirm button configured to confirm data migration or a cancel button configured to refrain from data migration. In some embodiments, the acquisition module 210 may receive a first acquisition request sent by a first device, wherein the first acquisition request carries a target account, the target account being obtained via a login interface of the first device; search, based on the target account and a first mapping relationship, for a preference setting corresponding to the target account, the first mapping relationship indicating a correlation between a plurality of accounts and a plurality of preference settings; designate the preference setting as a target preference setting corresponding to the target account, wherein the target preference setting includes a setting of one or more configuration options on a second device, the setting of the one or more configuration options being set by an operator on the second device using the target account, and the one or more configuration options including at least one of an inspection process configuration, a scanning scheme configuration, or a displaying configuration; send the target preference setting to the first device to facilitate the first device to update a setting corresponding to at least one configuration option on the first device based on the target preference setting; and start the first device. In some embodiments, the acquisition module 210 may obtain a first device identification of the first device and a second device identification of the second device, respectively; determine, based on the first device identification and the second device identification, whether the target preference setting is compatible with the first device; and in response to a determination that the target preference setting is incompatible with the first device, adjust the target preference setting; and update the setting corresponding to the at least one configuration option on the first device based on the adjusted target preference setting. In some embodiments, the acquisition module 210 may receive a second acquisition request sent by a first device, wherein the second acquisition request carries a target account, and the second acquisition request is sent by the first device based on a device identification of the second device; search, based on the target account and a second mapping relationship, for a preference setting corresponding to the target account, the second mapping relationship indicating correlations between a plurality of accounts and a plurality of preference settings; designate the preference setting as a target preference setting corresponding to the target account, wherein the target preference setting includes a setting of one or more configuration options on a second device, the setting of the one or more configuration options being set by an operator on the second device using the target account, and the one or more configuration options including at least one of an inspection process configuration, a scanning scheme configuration, or a displaying configuration; send the target preference setting to the first device to facilitate the first device to update a setting corresponding to at least one configuration option on the first device based on the target preference setting; and start the first device. In some embodiments, the acquisition module 210 may after one or more operators starts the second device using the plurality of accounts, for each of the plurality of accounts, receive a setting of one or more configuration options, to obtain the plurality of preference settings; and generate the second mapping relationship based on the correlations between the plurality of accounts and the plurality of preference settings. In some embodiments, the acquisition module 210 may send configuration data to a server, the configuration data including a device identification of the second device, at least one account, and at least one preference setting, the at least one account corresponding to the at least one preference setting one by one, each of the at least one preference setting including a setting of one or more configuration options on the second device, the setting of the one or more configuration options being set by an operator on the second device using a corresponding account, the setting of the one or more configuration options being configured to facilitate the server to generate a first mapping relationship based on the configuration data.

The imaging module 220 may be configured to perform an imaging operation using a first device based on the target configuration information. In some embodiments, the imaging module 220 may restart the first device after obtaining the target configuration information. In some embodiments, the imaging module 220 may determine, based on the twin data of the detection object, in the scanning scheme configuration, an initial image scanning scheme corresponding to the twin data of the detection object, the initial image scanning scheme including a plurality of scanning phases. In some embodiments, the imaging module 220 may execute the initial image scanning scheme; determine whether the portrait data of the detection object has changed during a current scanning phase of the plurality of scanning phases; and in response to a determination that the portrait data of the detection object has changed, optimize one or more parameters of at least one pending scanning phase of the plurality of scanning phases in the initial image scanning scheme. In some embodiments, the imaging module 220 may obtain historical scanning scheme data, the historical scanning scheme data including twin data of a historical object and/or twin data of a historical operator. In some embodiments, the imaging module 220 may fuse the historical scanning scheme data with at least a portion of the target configuration information to generate a target image scanning scheme for the detection object.

In some embodiments, the imaging module may include a first imaging module 222 and/or a second imaging module 224.

In some embodiments, the first imaging module 222 may obtain twin data of a detection object, the twin data of the detection object including portrait data of the detection object; determine, based on the twin data of the detection object, an initial image scanning scheme corresponding to the twin data of the detection object, the initial image scanning scheme including a plurality of scanning phases; execute the initial image scanning scheme; determine whether the portrait data of the detection object has changed during a current scanning phase of the plurality of scanning phases; in response to a determination that the portrait data of the detection object has changed, optimize one or more parameters of at least one pending scanning phase of the plurality of scanning phases in the initial image scanning scheme. In some embodiments, the first imaging module 222 may adjust at least one of a radio frequency (RF) pulse parameter, a gradient pulse parameter, or a signal acquisition time in the at least one pending scanning phase. In some embodiments, the first imaging module 222 may determine whether the changed portrait data of the detection object is related to an image scanning result; and in response to a determination that the changed portrait data of the detection object is related to the image scanning result, adjust the image scanning scheme. In some embodiments, the first imaging module 222 may determine whether the changed portrait data of the detection object includes information about an implant in the detection object; and in response to a determination that the changed portrait data of the detection object includes the information about the implant in the detection object, determine that the changed portrait data of the detection object is related to the image scanning result. In some embodiments, the first imaging module 222 may determine whether the changed portrait data of the detection object includes breath holding data of the detection object; and in response to a determination that the changed portrait data of the detection object includes the breath holding data of the detection object, determine that the changed portrait data of the detection object is related to the image scanning result. In some embodiments, the first imaging module 222 may fusing the portrait data of the detection object with twin data of an operator to generate the initial image scanning scheme corresponding to the twin data of the detection object. In some embodiments, the portrait data of the detection object may include at least one of morphological feature data of the detection object, inspection information of the detection object, or a physiological signal of the detection object. In some embodiments, the first imaging module 222 may perform a facial recognition on the detection object to obtain facial data of the detection object; and perform a query operation in a database based on the facial data to obtain the twin data of the detection object that matches with the facial data. In some embodiments, the first imaging module 222 may store target image data and a target image scanning scheme. The target image data may include image data obtained in each of the plurality of scanning phases; and the target image scanning scheme may be obtained by optimizing the one or more parameters of the at least one pending scanning phase.

In some embodiments, the second imaging module 224 may obtain historical scanning scheme data, the historical scanning scheme data including twin data of a historical object and twin data of a historical operator; obtain portrait data of a current object and initial twin data of a current operator; fuse the historical scanning scheme data with the portrait data of the current object and the initial twin data of the current operator to generate an image scanning scheme for the current object. In some embodiments, the second imaging module 224 may extract one or more historical scanning parameters from the twin data of the historical operator; and fuse the one or more historical scanning parameters with the portrait data of the current object and the initial twin data of the current operator to generate the image scanning scheme for the current object. In some embodiments, the second imaging module 224 may determine whether there is a correlation between the scanning ROI of the current object and the scanning ROI of the historical object; in response to a determination that there is a correlation between the scanning ROI of the current object and the scanning ROI of the historical object, obtain a portion of the twin data of the historical operator and all or a portion of the twin data of the historical object in the historical scanning scheme data; and fuse the portion of the twin data of the historical operator and the all or the portion of the twin data of the historical object in the historical scanning scheme data with the portrait data of the current object and the initial twin data of the current operator to generate the image scanning scheme for the current object. In some embodiments, the portion of the twin data of the historical operator may include at least one of a positioning manner, a scanning navigation parameter, a radio frequency (RF) parameter threshold, or a gradient parameter threshold. In some embodiments, the image scanning scheme for the current object and the historical scanning scheme data may be executed on different devices. In some embodiments, the second imaging module 224 may determine reference twin data of the historical object that matches with the portrait data of the current object in the historical scanning scheme data; determine reference twin data of the historical operator based on the reference twin data of the historical object; and fuse at least a portion of the reference twin data of the historical operator with the portrait data of the current object and the initial twin data of the current operator to generate the image scanning scheme for the current object. In some embodiments, the second imaging module 224 may obtain historical portrait data of the historical object from the twin data of the historical object; analyze data change of the portrait data of the current object compared with the historical portrait data to obtain an analysis result; and obtain, based on the analysis result, twin data of the historical object with a stability factor from the twin data of the historical object as the portion of the twin data of the historical object. In some embodiments, the twin data of the historical object with the stability factor in the twin data of the historical object may include at least one of body shape data of the historical object within a preset time period, implant information of the historical object within the preset time period, or an age of the historical object within the preset time period. In some embodiments, the second imaging module 224 may scan the current object using the image scanning scheme for the current object to generate an image scanning result; and store the image scanning result. The image scanning result may include twin data of the current object, imaging data of the current object, and/or twin data of the current operator.

It should be noted that the above descriptions of the system 200 and its modules is merely for convenience of description and may not limit the scope of the present disclosure. It could be understood that for the skills in this field, after understanding the principle of the system, various modules or form subsystems may be combined to connect with other modules without deviating from this principle. In some embodiments, the acquisition module 210 and the imaging module 220 disclosed in FIG. 2 may be different modules in the same system, or a module that implements the functions of two or more modules mentioned above. For example, each module may share a common storage module, and each module may also have its own storage module. Such deformations are within the scope of the present disclosure.

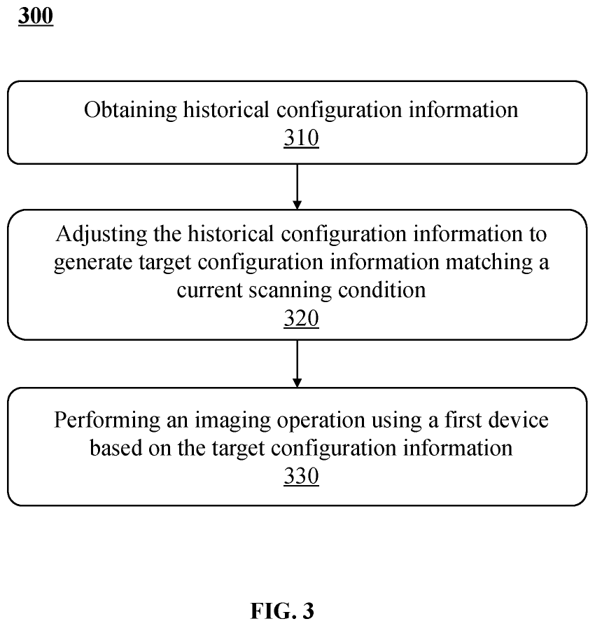

FIG. 3 is a flowchart an exemplary process of imaging according to some embodiments of the present disclosure. As shown in FIG. 3, process 300 may include one or more of the following operations. In some embodiments, the process 300 may be implemented by a processing device (e.g., the processing device 120).

In 310, historical configuration information may be obtained. Specifically, operation 310 may be executed by the acquisition module 210.

The historical configuration information may be information has been used to set a scanning device. In some embodiments, the historical configuration information may include at least one of a historical inspection process configuration, a historical scanning scheme configuration, a historical displaying configuration, and twin data of a historical detection object used when a historical scanning device is running in a historical scanning condition.

The historical inspection process configuration may refer to a historical configuration of an inspection process and/or an operation mode of the inspection process. In some embodiments, the historical inspection process configuration may include one or more of the following configurations: an inspection of an object status, an object positioning inspection, an inspection result, or the like.

For example, taking a head inspection process as an example, the historical inspection process configuration may include: an object registration (through scanning a quick response (QR) code or a bar code: radioiogy information system (RIS), automatically registering a height and/or a weight of the object, automatically recommending a scanning scheme); scanning (performing head flat scanning and enhancement automatically, triggering an enhancement automatically); image quality controlling (enabling a head motion monitoring); online image post-processing (performing a head vessel maximum intensity projection (MIP) automatically); film printing (performing a conventional operation automatically).

For example, taking a magnetic resonance imaging as an example, the historical inspection process configuration may include: determining whether the object is carrying a metal or metal implant; object positioning; scout image scanning; a cross-sectional T2 weighted image (t2_Jse_tra); a cross-sectional T1 weighted image (t1_tse_tra); a cross-sectional fat-suppressed T2 image (t2_trm_tra); a diffusion weighted image (ep2d_3scan_trace_tra); a sagittal T2_image (t2_Jse_sag).

For example, taking a CT inspection as an example, the historical inspection process configuration may include: obtaining a computed radiography (CR) image; determining an enhancement plan; turning on an intelligent mode with placing a monitoring line at the abdominal aorta level; clicking exposure confirmation; locating a detection line for scanning; injecting a contrast agent and monitoring the object; performing a first phase scanning; performing a second phase portal vein scanning.

For example, the historical inspection process configuration may include: automatically returning to a registration page or a main page when the inspection is completed; determining whether an operation process requires an operational guidance or determining which phase requires an operational guidance; checking whether a completed image needs to be automatically archived; checking whether the completed image needs to be marked with importance to remind the subsequent diagnosis and/or treatment process; checking whether the completed image is pushed to a post-processing application and waiting for the post-processing application to process the completed image; checking whether the completed image is automatically pushed to the background for preprocessing, or the like.

The historical scanning scheme configuration may refer to a historical configuration of a user scanning protocol and/or a collection scheme. The historical configuration of the user scanning protocol may be a historical configuration of a scanning protocol packet. In some embodiments, the historical configuration of the user scanning protocol may include a scanning protocol category, a scanning protocol execution order, a parameter setting of the scanning protocol in a fast file, and/or a parameter setting of the scanning protocol in a current scanning mode, etc. The scanning protocol category may reflect a requirement for a scanning image. For example, the scanning protocol category may reflect a quality requirement for the scanning image (e.g., a contrast that the scanning image needs to have), a specific type of the scanning image (e.g., T1WI, T2WI, fat suppression T2WI, etc.). The historical configuration of the collection plan may refer to a historical configuration of the collection plan content and/or a collection method. In some embodiments, the content of the collection plan may include, but may not be limited to: a collection site, a disease, a symptom, an object feature, etc. The collection method may be a collection technique for obtaining the scanning image. In some embodiments, the collection method may include, but may not be limited to an imaging technique (e.g., a fast spin echo (FSE) imaging, an echo planar imaging (EPI), a gradient recalled echo (GRE) imaging, etc.), a technology that meets a specific usage requirement (e.g., an acceleration technology, a mute technology, etc.) and a scanning technique (e.g., automatic/manual/custom, etc.).

The historical displaying configuration may be or include a configuration of a displaying technique and/or a display result. In some embodiments, the historical displaying configuration may include, but may not be limited to: an interface displaying style, a font size, multiple displayers, a page layout, whether remote connections being allowed, etc.

The twin data of the historical detection object may be projection data of a body state of the detection object in a virtual space. For detailed descriptions of the twin data of the historical detection object may be found elsewhere in the present disclosure, for example, relevant descriptions of operation 1310.

In some embodiments, the historical configuration information may be packaged in a second device and uploaded to a cloud server through the second device or a second mobile terminal. The second mobile terminal may be connected with or in communication with the second device.

In some embodiments, the acquisition module 210 may download the historical configuration information from the cloud server to a first device via the first device or a first mobile terminal. The first mobile terminal may be connected with or in communication with the first device.

In some embodiments, the historical configuration information may be packaged in the second device and stored in a storage medium or a third mobile terminal. In some embodiments, the acquisition module 210 may obtain the historical configuration information from the storage medium or the third mobile terminal via the first device.

In 320, the historical configuration information may be adjusted to generate target configuration information matching a current scanning condition. Specifically, operation 320 may be executed by the acquisition module 220.

The target configuration information may be information used to set a scanning device. Corresponding to the historical configuration information, in some embodiments, the target configuration information may include at least one of an inspection process configuration, a scanning scheme configuration, a displaying configuration, and twin data of a detection object. More description for the target configuration information may refer to the description for the historical configuration information.

In some embodiments, an account, a current scanning device, an operator, or a detection object under the current scanning condition is different from an account, the historical scanning device, an operator, or a detection object under the historical scanning condition, respectively.

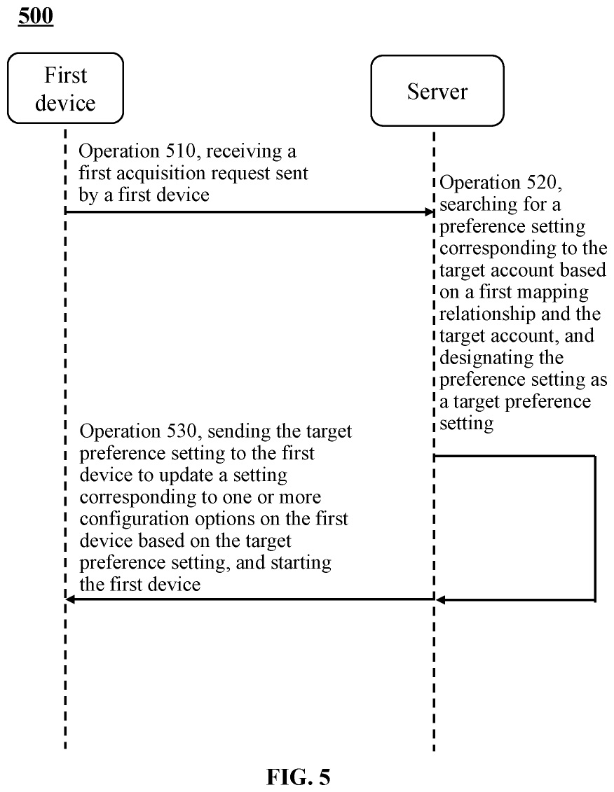

The detailed descriptions for obtaining the target configuration information may be found elsewhere in the present disclosure, for example, FIGS. 5-8 and the relevant descriptions.

In 330, an imaging operation is performed using a first device based on the target configuration information. Specifically, operation 330 may be executed by the acquisition module 230.

A target preference setting may include a setting of one or more configuration options on the second device. The setting of one or more configuration options may be set after a user starts the second device using a target account. The detailed descriptions for the target preference setting may be found elsewhere in the present disclosure, for example, operation 420 and the relevant descriptions.

The target account may be an account what the user using to start a scanning device. In some embodiments, the target account may match with the target configuration information. In some embodiments, the acquisition module 210 may obtain the target account. The detailed descriptions for the target account may be found elsewhere in the present disclosure, for example, operation 410 and the relevant descriptions.

In some embodiments, the imaging module 220 may update a setting of configuration option(s) on the first device based on the target preference setting in the target configuration information. The target preference setting may be related to the inspection process configuration, the scanning scheme configuration, and/or the displaying configuration in the target configuration information, or the like. The detailed descriptions for updating the setting of the configuration option(s) on the first device may be found elsewhere in the present disclosure, for example, FIGS. 5-8 and the relevant descriptions.

Furthermore, in some embodiments, the imaging module 220 may restart the first device. The first device may perform a scanning operation based on the updated configuration option(s).

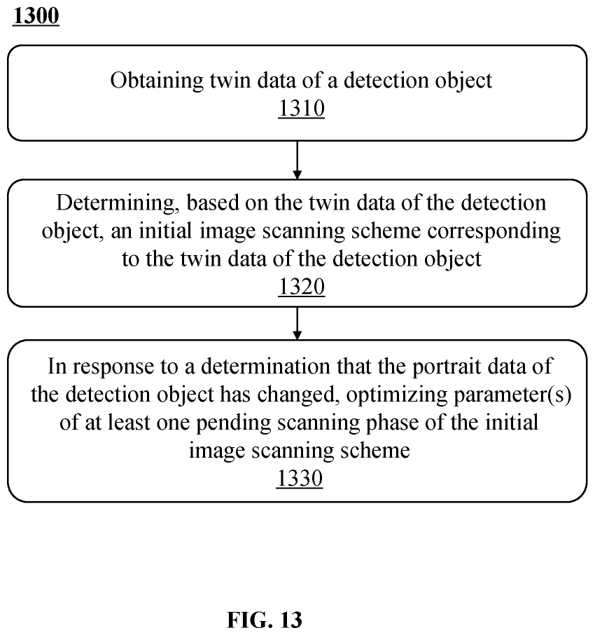

In some embodiments, a first imaging module 222 may perform an imaging operation using the first device based on the twin data of the detection object. Specifically, in some embodiments, the first imaging module 222 may determine initial image scanning scheme corresponding to the twin data of the detection object in the scanning scheme configuration based on the twin data of the detection object. The initial image scanning scheme may be divided into or include multiple scanning phases. Furthermore, in some embodiments, the first imaging module 222 may execute the initial image scanning scheme and determine whether the portrait data of the detection object has changed during a current scanning phase. In some embodiments, the first imaging module 222 may optimize one or more parameters of at least one pending scanning phase of the initial image scanning scheme in response to a determination that the portrait data of the detection object has changed. The detailed descriptions for performing the imaging operation using the first device based on twin data of the detection object may be found elsewhere in the present disclosure, for example, FIG. 13 and the relevant descriptions.

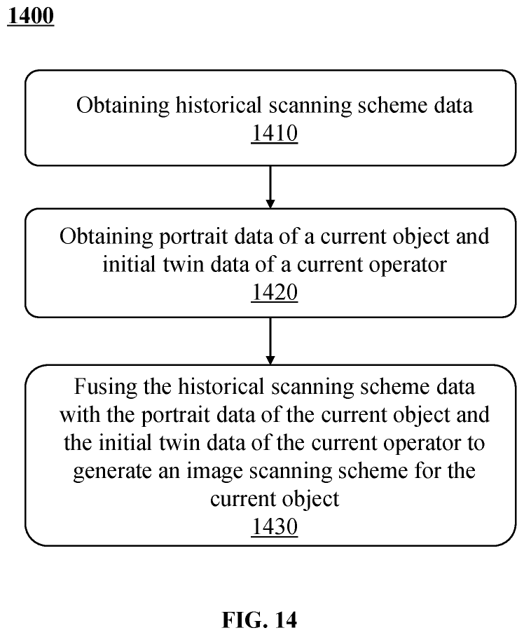

In some embodiments, a second imaging module 224 may utilize the first device to perform the imaging operation based on historical scanning scheme data. Specifically, in some embodiments, the second imaging module 224 may obtain the historical scanning scheme data. In some embodiments, the historical scanning scheme data may include twin data of a historical object and/or twin data of a historical operator. Furthermore, in some embodiments, the second imaging module 224 may fuse the historical scanning scheme data with at least a portion of the target configuration information to generate a image scanning scheme for a current object. The detailed descriptions for performing the imaging operation using the first device based on the historical scheme data may be found elsewhere in the present disclosure, for example, FIGS. 14-17 and the relevant descriptions.

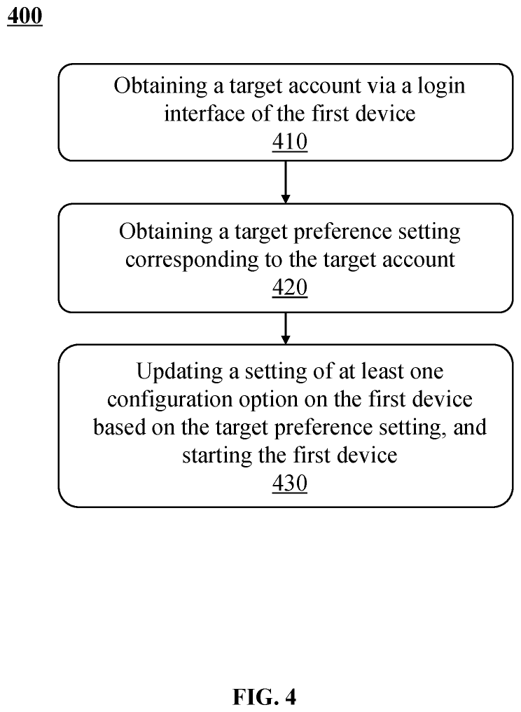

FIG. 4 is a flowchart illustrating an exemplary process for data migration implemented on a first device according to some embodiments of the present disclosure. As shown in FIG. 4, process 400 may include one or more of the following operations. In some embodiments, process 400 may be implemented by a processing device (e.g., the processing device 120) and/or the imaging system 200.

In 410, a target account input by a user may be received on a login interface of a first device.

The target account may be an account for the user to activate a scanning device. For example, the target account “A” may be an account for a user A to activate a second device.

In some embodiments, the first device may receive the target account input by the user on the login interface of the first device. For example, the first device may receive the target account “A” input by the user A on the login interface of the first device.

In some embodiments, the first device may pop up a data migration pop-up window in response to a login operation triggered by the user on the login interface of the first device. In some embodiments, a manner through which a user triggers the login operation on the login interface of the first device may include, but may not be limited to, entering a correct target account, entering a correct password corresponding to the target account, clicking a login button, performing a fingerprint recognition operation, performing a facial recognition operation, or the like, or any combination thereof.

In some embodiments, the data migration pop-up window may include a confirm button configured to confirm data migration or a cancel button configured to refrain from data migration. In some embodiments, if the user clicks the confirm button (indicating that the user wants to implement the data migration function), the operation 420 for obtaining a target preference setting may be executed. If the user clicks the cancel button (indicating that the user does not want to implement the data migration function), the first device may be started directly without obtaining the target preference setting.

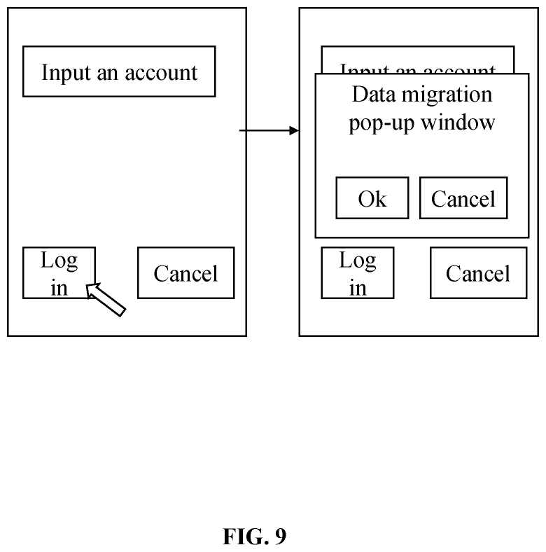

For example, if the user A needs to use the first device, a power button of the first device may be clicked on. After the first device detects the user's click on the power button, the login interface of the first device may be displayed. For example, FIG. 9 is a schematic diagram of an exemplary login interface of the first device according to some embodiments of the present disclosure. As shown in FIG. 9, the login interface of the first device is equipped with an account input box, a login button, and a cancel button. The user may input the target account in the account input box, and then click the login button. After the first device detects the user's click on the login button, a situation that the user has triggered the login operation may be determined. In response to the login operation triggered by the user on the login interface of the first device, the first device may pop up the data migration pop-up window to ask whether the user wants to migrate data to the first device. The data migration pop-up window may include the confirm button and the cancel button. If the user wants to migrate data from other device(s) to the first device, the confirm button may be clicked. If the user does not want to migrate data to the first device, the cancel button may be clicked.

In the above embodiment, the design of the login interface and the data migration pop-up window may allow the user to input the target account for subsequent data migration. The above inputting manner is simple and fast, and the user experience is improved.

In 420, a target preference setting corresponding to the target account may be obtained.

The target preference setting may include a setting of one or more configuration options on a second device. The setting of one or more configuration options may be set after the user starts the second device using the target account.

In some embodiments, the configuration option(s) may include at least one of the inspection process configuration, the scanning scheme configuration, and the displaying configuration, etc. The detailed descriptions for the inspection process configuration, scanning scheme configuration, and displaying configuration may be found elsewhere in the present disclosure, for example, operation 310 and the relevant descriptions. In some embodiments, the configuration option(s) may also include operation mode configuration. For example, the operation mode configuration may include the following option(s) in object registration: whether the user manually fills a box with a weight of the object or manually selects a plan, or a system automatically recognizes the weight of the object or recommends a plan.

In some embodiments, after the user sets the configuration option(s) on the second device, the second device may send a correlation between the target account used by the user and a setting result, and/or a correlation between the target account used by the user, the setting result, and a device identification of the second device to a server. The first device may obtain the target preference setting via an interaction with the server.

Specifically, the first device may send a first acquisition request to the server. The first acquisition request may be sent by the first device to the server, and the first acquisition request may include obtaining the target preference setting corresponding to the target account. In some embodiments, the first acquisition request may carry the target account, such as the target account “A”.

Furthermore, the server may search for the corresponding preference setting based on a first mapping relationship and the target account, and use the preference setting as the target preference setting. In some embodiments, the first mapping relationship may indicate a correlation between a plurality of accounts and a plurality of preference settings. The detailed descriptions for the target preference setting obtained by the server may be found elsewhere in the present disclosure, for example, FIG. 5 and the relevant descriptions.

Furthermore, the first device may receive the target preference setting sent by the server.

In some embodiments, after the user sets the configuration option(s) on the second device, the second device may save the correlation between the target account used by the user and the setting result, and/or the correlation between the target account used by the user, the setting result, and the device identification of the second device. The first device may obtain the target preference setting through the interaction with the second device.

Specifically, after the first device obtains the target account, a second acquisition request may be sent to the second device based on the device identification of the second device by the first device. The second acquisition request may be a request sent by the first device to the second device. The second acquisition request may be a request for obtaining the target preference setting corresponding to the target account. In some embodiments, each device may store a mapping table between the device identifications and IP addresses. After the first device obtains the target account and the device identification of the second device, a corresponding IP address of the second device may be searched based on the device identification of the second device, and the second acquisition request may be sent to the second device based on the corresponding IP address.

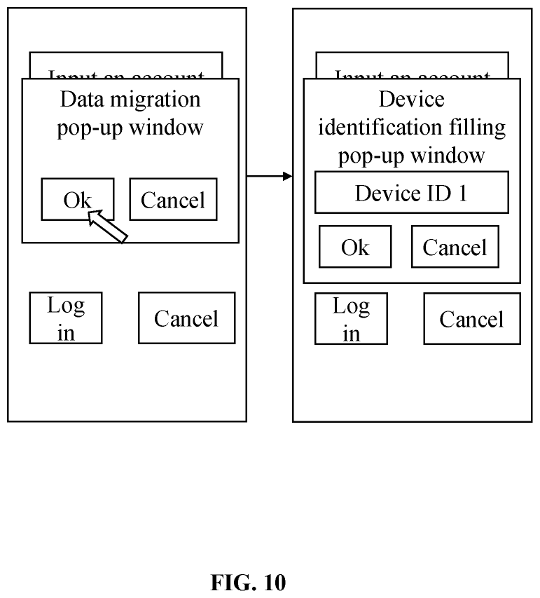

In some embodiments, the second acquisition request may carry the target account. For example, FIG. 10 is a schematic diagram illustrating an exemplary interface for inputting a device identification of a first device according to some embodiments of the present disclosure. As shown in FIG. 10, after filling in a device identification filling pop-up window with the device identification “Device ID 1” of the second device, the user may click the confirm button in the pop-up window to fill in the device identification. After the first device detects the click operation, the second acquisition request may be sent with the target account “A” to the second device.

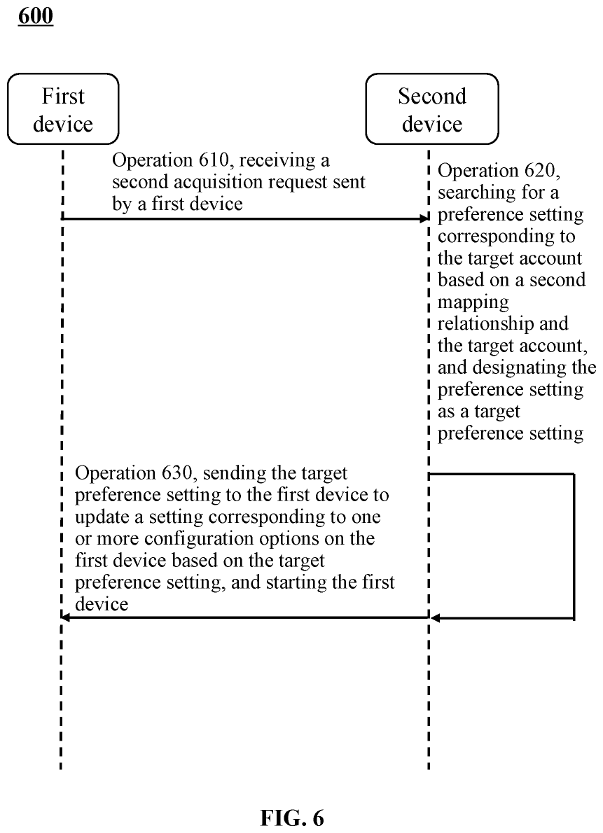

Furthermore, the second device may search for a corresponding preference setting based on the second mapping relationship and the target account, and use the corresponding preference setting as the target preference setting. The detailed descriptions for the target preference setting obtained by the second device may be found elsewhere in the present disclosure, for example, FIG. 6 and the relevant descriptions.

Furthermore, the first device may receive the target preference setting sent by the second device.

In some embodiments, after the user sets the configuration option(s) on the second device, the second device may send the correlation between the target account used by the user and the setting result, and/or the correlation between the target account used by the user, the setting result, and the device identification of the second device to a terminal. The first device may obtain the target preference setting via an interaction with the terminal.

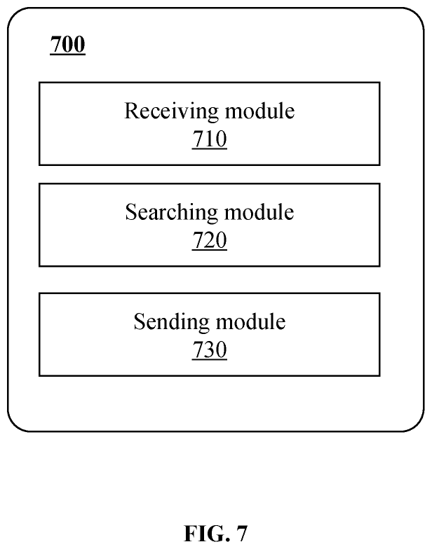

The detailed descriptions for obtaining the target preference setting by the first device via the interaction with the terminal may be found elsewhere in the present disclosure, for example, FIG. 7 and the relevant descriptions.

In some embodiments, the target preference setting of the second device may not be compatible with the first device, so the first device may adjust the target preference setting and update the setting of the configuration option(s) on the first device based on the adjusted target preference setting.

Specifically, in some embodiments, the first device may obtain the device identification of the second device. In some embodiments, in response to a triggering operation for the confirm button triggered by the user in the data migration pop-up window, the first device may pop up a device identification filling pop-up window, and the device identification filling pop-up window may be used to facilitate the user to input the device identification. Then the first device may receive the device identification of the second device input by the user in the pop-up window. For example, as shown in FIG. 10, after the user clicks the confirm button in the data migration pop-up window, the first device may pop up the device identification filling pop-up window. The user may input the device identification “Device ID 1” of the second device in the device identification filling pop-up window, and then click the confirm button.

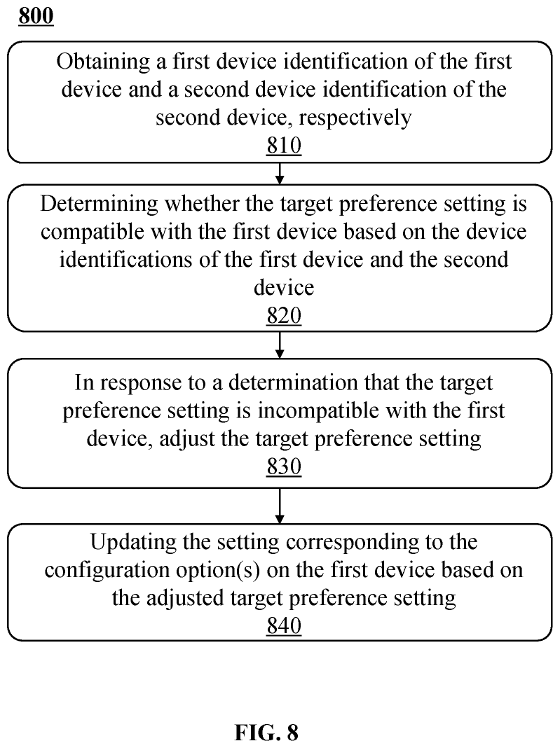

Furthermore, in some embodiments, the first device may determine whether the target preference setting is compatible with the first device based on the device identification of the second device; adjust the target preference setting in response to a determination that the target preference setting is not compatible with the first device; and update a setting of one or more configuration options on the first device based on the adjusted target preference setting. The detailed descriptions for updating the setting of the configuration option(s) on the first device may be found elsewhere in the present disclosure, for example, FIG. 8 and the relevant descriptions.

In 430, the setting of the configuration option(s) on the first device may be updated based on the target preference setting, and the first device may be started.

For example, the configuration option(s) on the first device may include the inspection process configuration, scanning scheme configuration, and/or displaying configuration, etc. The target preference setting may include a result for setting the configuration option of “inspection process configuration”, a result for setting the configuration option of “scanning scheme configuration”, and a result for setting the configuration option of “displaying configuration” determined by the user. After the first device obtains the target preference setting, the setting corresponding to the configuration option of “inspection process configuration” on the first device may be updated based on the user's setting for the configuration option of “inspection process configuration”; the setting corresponding to the configuration option of “scanning scheme configuration” on the first device may be updated based on the user's setting for the configuration option of “scanning scheme configuration”; and the setting corresponding to the configuration option of “displaying configuration” on the first device may be updated based on the user's setting for the configuration option of “displaying configuration”. After the updating operation is completed, the first device may be started.

It should be understood that the “start” in the embodiments of the present disclosure may also be understood as “login”, and starting the first device may be understood as opening the homepage of the examination system installed on the first device. The user may start to perform corresponding inspections on an object based on content on the homepage.

As described in a data migration method provided by the embodiments of the present disclosure, the target account may be obtained via the login interface of the first device, which may be used to subsequently migrate the configuration data on the second device to the first device. Then the target preference setting that matches with the target account may be obtained. The target preference setting may be the result of setting configuration option(s) on the second device set after the user starts the second device using the target account. Based on the target preference setting, the setting corresponding to the configuration option(s) on the first device may be updated and the first device may be started. The entire process above only requires the user to input the target account, and the result of setting configuration option(s) on the second device may be migrated to the first device. There is no need for the user to make another configuration on the first device, thus saving the user workload and improving the user experience.

FIG. 5 is a flowchart illustrating an exemplary process for data migration implemented on a server according to some embodiments of the present disclosure. As show in FIG. 5, process 500 may include one or more of the following operations. In some embodiments, process 500 may be implemented by a processing device (e.g., the processing device 120).

In 510, a first acquisition request sent by a first device may be received.

The detailed descriptions for the first acquisition request may be found elsewhere in the present disclosure, for example, operation 420 and the relevant descriptions.

In some embodiments, the first acquisition request may carry a target account. In some embodiments, the target account may be obtained via a login interface of the first device. The detailed descriptions for the target account may be found elsewhere in the present disclosure, for example, operation 410 and the relevant descriptions.

In 520, a preference setting corresponding to the target account may be searched based on a first mapping relationship and the target account, and the preference setting may be designated as a target preference setting.

The first mapping relationship may indicate a correlation between a plurality of accounts and a plurality of preference settings. The preference setting may include setting one or more configuration options on a second device, the setting of the one or more configuration options being set by an operator the second device using a corresponding account. For example, the preference setting may be the result of setting the configuration option(s) set after the user starts the second device using an account A, that is, the preference setting may be preference setting A.

In some embodiments, the server may generate the first mapping relationship based on configuration data sent by the second device. The detailed descriptions for the configuration data sent by the second device to the server may be found elsewhere in the present disclosure, for example, operation 620 and the relevant descriptions.

It could be understood that a scanning device may be used by multiple users. After using an account to start the scanning device, a user may set the configuration option(s). Therefore, the configuration data sent by the second device to the server may include at least one account, at least one preference setting, at least one device identification of the second device, or the like, in which there is a one-to-one correspondence between the at least one account, the at least one preference setting, and the at least one device identification of the second device.

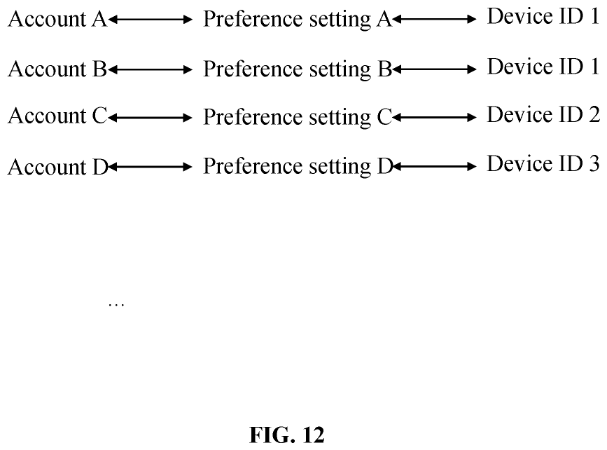

For example, FIG. 12 is a schematic diagram of the first mapping relationship according to some embodiments of the present disclosure. As shown in FIG. 12, a plurality of accounts (e.g., Account A, Account B, Account C, Account D, . . . ) may have a one-to-one correspondence with a plurality of preference settings (e.g., Preference A, Preference B, Preference C, Preference D, . . . ) and device identifications (e.g., Device ID 1, Device ID 2, Device ID 3, . . . ) in the first mapping relationship. The same device identification may correspond to the same second device. For example, the Preference settings A and B corresponding to Device ID 1 may be settings of configuration option(s) for any user using Account A and Account B on the corresponding second device. Different device identifications may correspond to different second devices. For example, the Preference setting C corresponding to device ID 2 may be a setting of configuration option(s) for any user using Account C on a second device, while the Preference setting D corresponding to device ID 3 may be a setting of configuration option(s) for any user using Account D on another second device.

Based on the descriptions in operation 420, the target preference setting may be the result of setting the configuration option(s) on the second device set after the user starts the second device using the target account. In some embodiments, the configuration option(s) may include at least one of the inspection process configuration, the scanning scheme configuration, and the displaying configuration.

Merely by way of example, as shown in FIG. 12, assuming that in the first acquisition request received by the server, the target account is Account A, and the device identification of the second device is Device ID 1, the corresponding Preference setting A may be searched based on the first mapping relationship shown in FIG. 12, then the Preference setting A may be used as the target preference setting.

In some embodiments, the target preference setting of the second device may be incompatible with the first device, and the server may adjust the target preference setting and update the setting corresponding to the configuration option(s) on the first device based on the adjusted target preference setting. The detailed descriptions for updating the configuration option(s) may be found elsewhere in the present disclosure, for example, FIG. 8 and the relevant descriptions.

In 530, the target preference setting may be sent to the first device to update a setting corresponding to one or more configuration options on the first device based on the target preference setting. In some embodiments, the first device may be started.

The detailed descriptions for updating the configuration option of the first device may be found elsewhere in the present disclosure, for example, operation 430 and the relevant descriptions.

For example, the server may send the preference setting A as the target preference setting to the first device.

In some embodiments of the present disclosure, the first device may obtain the target preference setting via an interaction with the server. Specifically, for a certain device, the user may start the device using the user's account and complete the setting of configuration option(s) on the device. The device may send the configuration data to the server, enabling the server to establish the first mapping relationship based on the received configuration data. After the server receives the request sent by the first device, the target preference setting may be searched from the first mapping relationship, and then returned to the first device, allowing the user to migrate a configuration made on the second device to the first device without the need for the user to make another configuration on the first device, saving a lot of workload and improving the user experience.

FIG. 6 is a flowchart illustrating an exemplary process for data migration implemented on a second device according to some embodiments of the present disclosure. As shown in FIG. 6, process 600 may include one or more of the following operations. In some embodiments, process 600 may be implemented by a processing device (e.g., the processing device 120).

In 610, a second acquisition request sent by a first device may be received.

The second acquisition request may be sent via the first device based on a device identification of a second device. The detailed descriptions for the second acquisition request may be found elsewhere in the present disclosure, for example, operation 420 and the relevant descriptions.

In some embodiments, the second acquisition request may carry a target account. The detailed descriptions for the target account may be found elsewhere in the present disclosure, for example, operation 410 and the relevant descriptions.

In 620, a preference setting corresponding to the target account may be searched based on a second mapping relationship and the target account, and the preference setting may be designated as a target preference setting.

The second mapping relationship may indicate a correlation between a plurality of accounts and a plurality of preference settings. In some embodiments, after one or more operators starts the second device using the plurality of accounts, for each of the plurality of accounts, the second device may receive a setting of one or more configuration options, to obtain a plurality of corresponding preference settings.

Merely by way of example, a homepage setting of a medical examination system includes a setting portal of configuration option(s), which may allow the user to open a setting interface of the configuration option(s). The user may set the configuration option(s) via the setting interface. For example, after using the user's account A to start a device (e.g., the second device), user A may open the setting interface of the configuration option(s). The setting interface of the configuration option(s) may include one or more configuration options: inspection process configuration, scanning scheme configuration, and/or displaying configuration. The user may set these configuration options separately to obtain the corresponding preference setting A. Similarly, the second device may obtain the preference setting B corresponding to account B. After the second device obtains the preference setting, the correlation between the plurality of accounts and the plurality of preference settings used by the users for starting the second device may be stored.

In some embodiments, the second device may generate the second mapping relationship based on the correlation between the plurality of accounts and the plurality of preference settings. It should be understood that the second device may be used by multiple users, and the multiple users may have set the configuration options after starting the second device using their accounts. Therefore, the second device may obtain multiple groups of correlations, and the second device may generate the second mapping relationship based on the multiple groups of correlations.

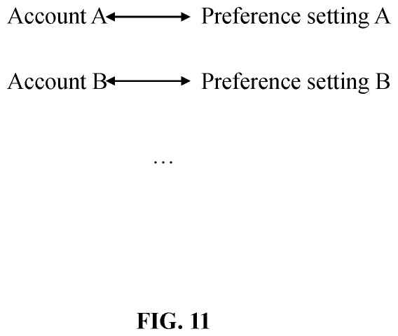

For example, FIG. 11 is a schematic diagram of the second mapping relationship according to some embodiments of the present disclosure. As shown in FIG. 11, the second device may generate the second mapping relationship based on the correlation between “Account A” and “Preference setting A”, “Account B” and “Preference setting B”, etc.

In some embodiments, the second device may further send configuration data to a server, allowing the server to generate a first mapping relationship based on the configuration data. The detailed descriptions for the first mapping relationship may be found elsewhere in the present disclosure, for example, operation 520 and the relevant descriptions.

In some embodiments, the configuration data may include a device identification of the second device, at least one account, and at least one preference setting, or the like. The detailed descriptions for the preference setting may be found elsewhere in the present disclosure, for example, operation 520 and the relevant descriptions.

Merely by way of example, if the device identification of the second device is “Device ID 1”, then the second device may send “Account A—Preference A—Device ID 1”, “Account B—Preference B—Device ID 1”, . . . to the server.

Based on the descriptions in operation 420, the target preference setting may be the result of setting the configuration option(s) on the second device set after the user starts the second device using the target account. In some embodiments, the configuration option(s) may include at least one of inspection process configuration, scanning scheme configuration, and displaying configuration, etc.

As shown in FIG. 11, assuming that the second device receives the second acquisition request and the target account is an account A, and the corresponding preference setting is searched based on the first mapping relationship as a preference setting A, then the preference setting A may be used as the target preference setting.

In 630, the target preference setting may be sent to the first device to update a setting corresponding to the configuration option(s) on the first device based on the target preference setting. In some embodiments, the first device may be started.

The detailed descriptions for the first device updating the configuration option(s) based on the target preference setting may be found elsewhere in the present disclosure, for example, operation 430 and the relevant descriptions. In some embodiments, after updating the configuration option(s), the first device may be started based on the updated configuration option(s).

In some embodiments of the present disclosure, the first device may obtain the target preference setting via an interaction with the second device. For the second device, after the user starts the second device with different accounts, for each account, the second device may receive the result of the user setting the configuration option(s) and obtain the preference setting; a second mapping relationship may be generated based on the correlation between the plurality of accounts and the plurality of preference settings. After the second device receives the request sent by the first device, the target preference setting may be searched from the second mapping relationship, and then the target preference setting may be returned to the first device, allowing users to migrate the configuration made on the second device to the first device without the need for users to make another configuration on the first device, saving a lot of workload and improving the user experience.

FIG. 7 is a block diagram illustrating an exemplary terminal for data migration according to some embodiments of the present disclosure. In some embodiments, a terminal may include a first mobile terminal and/or a second mobile terminal. The first mobile terminal may be connected with a first device. The second mobile terminal may be connected with a second device.

As shown in FIG. 7, the terminal (the first mobile terminal and/or the second mobile terminal) may include a receiving module, a searching module, and/or a sending module.

The receiving module may be used to receive a second acquisition request sent by the first device. In some embodiments, the second acquisition request may carry a target account. The second acquisition request may be sent via the first device based on a device identification of the second device. The detailed descriptions for receiving the second acquisition request sent by the first device may be found elsewhere in the present disclosure, for example, operation 610 and the relevant descriptions.

The searching module may be used to search for a preference setting corresponding to the target account based on a second mapping relationship and the target account, and designate the preference setting as a target preference setting. The second mapping relationship may indicate a correlation between the plurality of accounts and the plurality of preference settings. The target preference setting may be a result of setting one or more configuration options on the second device set after a user starts the second device using the target account. In some embodiments, the configuration option may include at least one of the inspection process configuration, scanning scheme configuration, and displaying configuration, etc. The detailed descriptions for searching for the preference setting corresponding to the target account based on the second mapping relationship and the target account and designating the preference setting as the target preference setting may be found elsewhere in the present disclosure, for example, operation 620 and the relevant descriptions.

The sending module may send the target preference setting to the first device to update a setting corresponding to configuration option(s) on the first device based on the target preference setting and start the first device. The detailed descriptions for sending the target preference setting to the first device to update a setting corresponding to configuration option(s) on the first device based on the target preference setting and starting the first device may be found elsewhere in the present disclosure, for example, operation 630 and the relevant descriptions.

FIG. 8 is a flowchart illustrating an exemplary process for updating setting(s) of configuration option(s) on a first device according to some embodiments of the present disclosure. As shown in FIG. 8, process 800 may include one or more of the following operations. In some embodiments, process 800 may be implemented by a processing device (e.g., the processing device 120).

In 810, device identifications of a first device and a second device (e.g., a first device identification of the first device and a second device identification of the second device) may be obtained, respectively.

In some embodiments, a first acquisition request sent by the first device and received by a server may include the device identification of the first device. Specifically, the server may obtain the device identification of the first device based on the first acquisition request, such as “Device ID 3”.

Based on the descriptions in operations 520, the server may store an account, a device identification, and a preference setting used by a user for starting the second device. As shown in FIG. 12, there may be a one-to-one correspondence between accounts, device identifications, and preference settings.

In some embodiments, the server may obtain the device identification of a corresponding second device based on the target account in the first acquisition request. For example, as shown in FIG. 12, the server may obtain a device identification “Device ID 1” of the corresponding second device based on the “Target account A” in the first acquisition request.

In 820, whether the target preference setting is compatible with the first device may be determined based on the device identifications of the first device and the second device.

In some embodiments, the server may determine a model, a serial number, a name, etc. of the second device based on the device identification of the second device. In some embodiments, the server may also obtain intelligent algorithms corresponding to various configuration options in the target preference settings. In either of the following situations, it should be considered that the target preference setting is incompatible with the first device, including: models of the first devices and the second device are different; serial numbers of the first device and the second device are different; the serial numbers of the first device and the second device are the same, but names of the first device and the second device are different; the first device and the second device have the same model, but the first device does not support an intelligent algorithm corresponding to a configuration option in the target preference setting. In other situations beyond the above situations, it may be considered that the target preference setting is compatible with the first device.

In 830, in response to a determination that the target preference setting is incompatible with the first device, the target preference setting may be adjusted.

In some embodiments, if the target preference setting is incompatible with the first device, the server may display a prompt message to remind the user that the first device and target preference setting are incompatible, allowing the user to adjust the target preference setting based on a setting supported by the first device to obtain the adjusted target preference setting. For example, a displayer corresponding to “displaying configuration” in the target preference setting may be set as “multiple displayers tiled for displaying”. If the first device only has one displayer, the server may display a prompt message, and the user may adjust the “displaying configuration” to “single player for displaying”.

In some embodiments, if the models of the first device and the second device are the same, but the first device does not support the intelligent algorithm corresponding to a certain configuration option in the target preference settings, incompatible items and reasons may be listed before or after data migration, and the user may confirm subsequent operations (e.g., removing incompatible items, adaptively optimizing, etc.). Continuing with the above example, the server may send a prompt to the user with massage “The first device only contains one display, please confirm whether to adjust the display configuration”, and wait for the user to confirm before completing an adjustment operation.

In some embodiments, if the serial numbers of the first device and the second device are the same, but the names of the first device and the second device are different, a comparison table may be built-in in a system, the serial numbers and/or the names of the first device and/or the second device may be converted based on the comparison table, or one or more incompatible sequences may be provided, and a comparison relationship may be set by the user.

In 840, the setting corresponding to the configuration option(s) on the first device may be updated based on the adjusted target preference setting.

Specifically, in some embodiments, the server may send the adjusted target preference setting to the first device, allowing the first device to update the setting corresponding to the configuration option(s) based on the adjusted target preference setting (e.g., “single displayer for displaying”).

In some embodiments of the present disclosure, the server may determine whether the target preference setting is compatible with the first device based on the device identifications of the first device and the second device. In incompatible situations, the server may adjust the target preference setting and update the setting corresponding to the configuration option(s) on the first device based on the adjusted target preference setting. By considering the compatibility between the preference settings and devices, a data migration scheme may be more diversified.

FIG. 13 is a flowchart illustrating an exemplary process for optimizing an image scanning scheme based on twin data of a detection object according to some embodiments of the present disclosure. As shown in FIG. 13, process 1300 may include one or more of the following operations. In some embodiments, process 1300 may be implemented by a processing device (e.g., the processing device 120) and/or a imaging system 200 (e.g., the first imaging module 222).

In 1310, twin data of a detection object may be obtained.

The twin data of the detection object may be projection data of a physical state of the detection object in a storage space. For example, the twin data of the detection object may be the projection data of the physical state of the detection object stored in the storage device 150. In some embodiments, the twin data of the detection object may include portrait data of the detection object.

The portrait data of the detection object may be a group of feature(s) and/or relevant information of the detection object. In some embodiments, the portrait data of the detection object may include at least one of morphological feature data of the detection object, inspection information of the detection object, and a physiological signal of the detection object, etc.

The morphological feature data of the detection object may be or include data related to an external morphology and basic information of the detection object. In some embodiments, the morphological feature data of the detection object may include a facial feature, a height, a weight, a gender, an age, implant information, pregnancy, health status, etc. The health status may include whether the detection object is weak, whether a wheelchair is needed, whether a transport bed is needed, whether the detection object is unconscious, and whether the detection object has any trauma. In some embodiments, the morphological feature data of the detection object may be obtained from a database by manually entering the database in an admission registration system. In some embodiments, the admission registration system may input the facial image of the detection object into the database for storage.

The inspection information of the detection object may be or include diagnostic information and/or physical inspection information of the detection object. In some embodiments, the inspection information of the detection object may include an outpatient diagnosis result (e.g., a fever, a pain, an unconsciousness, a regularity of breathing) from a doctor, a scanning image and a scanning image diagnosis result, blood test reports, etc. In some embodiments, the inspection information of the detection object may be obtained from a database by manually inputting in the medical system or other methods.

The physiological signal of the detection object may be a real-time monitoring physiological signal of the detection object. In some embodiments, the physiological signal of the detection object may include, but may not be limited to a cardiac cycle, a respiratory cycle, etc. In some embodiments, the physiological signals of the detection object may be obtained automatically from a database via a hospital bed, an aperture sensor, and a health monitoring equipment.

In some embodiments, the twin data of the detection object may include data such as an inspection site, which may not be limited in the present disclosure.

In some embodiments, the first imaging module 222 may perform a facial recognition on the detection object to obtain facial data of the detection object. The facial data of the detection object may include at least one facial image of the detection object. In some embodiments, the first imaging module 222 may perform a query operation in a database based on the facial data to obtain twin data of the detection object that matches with the facial data. Specifically, the first imaging module 222 may extract a plurality of image features from at least one facial image of the detection object, and match the plurality of image features with image features of the facial image stored in the database. The successfully matched facial image may be used as the matched facial data, and the twin data of the detection object that matches with the facial data may be obtained.

In some embodiments of the present disclosure, the facial recognition may be performed on the detection object to obtain the facial data of the detection object, and then the facial data may be queried in the database to obtain the twin data of the detection object that matches with the facial data, thus ensuring the accuracy of the twin data of the detection data.

In 1320, an initial image scanning scheme corresponding to the twin data of the detection object may be determined based on the twin data of the detection object.

The initial image scanning scheme may be an unexecuted image scanning scheme.

In some embodiments, the initial image scanning scheme may be divided into or include a plurality of scanning phases. For example, taking a cardiac magnetic resonance imaging as an example, the initial image scanning scheme may be divided into: a cardiac transverse scanning phase; a cardiac rough two-chamber scanning phase; a cardiac four-chamber scanning phase; a cardiac short axis scanning phase, etc.

In some embodiments, the first imaging module 222 may determine the initial image scanning scheme based on the twin data of the detection object. For example, the first imaging module 222 may select an abdominal breath holding or a free breath acquisition scheme based on a breathing state of the detection object, and determine a duration of each breath holding based on a breath holding ability of the detection object. For example, the first imaging module 222 may automatically select a collection scheme with a metal artifact suppression based on the implant information of the detection object. For example, the first imaging module 222 may use a physiological structure loss (e.g., a congenital variation, or an acquired surgery) of the detection object as an input of an image analysis algorithm in the initial image scanning scheme, thereby avoiding erroneous analysis results.

In some embodiments, the first imaging module 222 may query a historical object database based on the portrait data of the detection object to obtain the best practical solution as the initial image scanning scheme that matches with the twin data of the detection object. For example, the first imaging module 222 may match twin data of a historical object in the historical object database with twin data of a current detection object, and uses the practice scheme with the highest matching degree corresponding to the historical object as the initial image scanning scheme. The detailed descriptions of the twin data of historical object(s) may be found elsewhere in the present disclosure, for example, FIG. 14 and the relevant descriptions.

Twin data of an operator (e.g., a technician) may be projection data of preference settings of operator's operations in a storage space. In some embodiments, the acquisition module 210 may obtain the twin data of the operator from a second device via a target account of the operator. Specifically, the acquisition module 210 may use an account used by the operator as the target account and obtain, from the second device, a target preference setting that matches with the target account as the twin data of the operator. The detailed descriptions for obtaining the target preference setting that matches with the target account may be found elsewhere in the present disclosure, for example, FIGS. 4-8 and the relevant descriptions.

In some embodiments, the first imaging module 222 may fuse the portrait data of the detection object with the twin data of the operator to generate an initial image scanning scheme corresponding to the twin data of the detection object. For example, the first imaging module 222 may select a type of a scanning protocol package based on the twin data of the operator. For example, the first imaging module 222 may select a displaying manner for a scanning image based on the twin data of the operator.

In some embodiments, the first imaging module 222 may search for multiple executed scanning schemes in a historical detection object database based on the portrait data of the detection object, and further automatically select the practice scheme with the highest matching degree between twin data of the multiple historical operators and twin data of the current operator from the multiple executed scanning schemes as the initial image scanning scheme. The detailed descriptions for the twin data of the historical operator may be found elsewhere in the present disclosure, for example, FIG. 14 and the relevant descriptions.

For example, the first imaging module 222 may match twin data of a historical detection object in the historical detection object database with twin data of a current detection object, obtain 5 practice schemes corresponding to the historical detection objects with the top 5 matching degrees between the twin data of historical operators and twin data of the current operator, and then select a practice scheme with the highest matching degree from the 5 practice schemes as the initial image scanning scheme.

In 1330, in response to a determination that the portrait data of the detection object has changed, parameter(s) of at least one pending scanning phase of the initial image scanning scheme may be optimized.

In some embodiments, the first imaging module 222 may determine whether the changed portrait data of the detection object is related to a image scanning result.

In some embodiments, the first imaging module 222 may determine whether the changed portrait data of the detection object includes information about an implant in the detection object. If the changed portrait data of the detection object include the information about an implant in the detection object, the changed portrait data of the detection object is related to the image scanning result. The information about the implant in the detection object may include whether there is an implant in the body of the detection object, and information about a position, a shape, a size, and/or a material of the implant. Merely by way of example, if a metal implant is added into the body of the detection object, the portrait data of the detection object may be related to the image scanning result.

In some embodiments, the first imaging module 222 may determine whether the changed portrait data of the detection object includes breath holding data of the detection object. If the changed portrait data of the detection object includes the breath holding data of the detection object, the changed portrait data of the detection object may be related to the image scanning result. The breath holding data of the detection object may include a breath holding duration of the detection object. Merely by way of example, if the breath holding duration of the detection object is shortened, the changed portrait data of the detection object may be related to the image scanning result.

In some embodiments of the present disclosure, the first imaging module 222 may determine whether the portrait data of the detection object has changed, and further determine whether the changed portrait data of the detection object is related to the image scanning result. Only when both conditions are met, the image scanning scheme may be adjusted, thus further improving the accuracy of a recommended image scanning scheme and ensuring the accuracy of an inspection result.

In some embodiments, if it is determined that the changed portrait data of the detection object is related to the image scanning result, the image scanning scheme may be adjusted.

Specifically, in some embodiments, the first imaging module 220 may adjust radio frequency (RF) pulse parameter(s), gradient pulse parameter(s), and a signal acquisition time during the pending scanning phase.

The RF pulse parameter(s) may include a bandwidth of the RF pulse (i.e., a frequency range of the RF pulse), an amplitude of the RF pulse (i.e., an intensity of the RF pulse), a time to apply the RF pulse, a duration of the RF pulse, etc. The gradient pulse parameter(s) may include a direction of a gradient field, a field strength of the gradient field, a time to apply the gradient field, a duration of the gradient field, etc. The signal acquisition time may include a time to collect the signal. A setting of parameters related to scanning and arrangement of the parameters in a time sequence may be referred to as an inspection protocol, and the parameters may include a pulse, a gradient field, a signal acquisition time, etc.

In some embodiments, the first imaging module 220 may achieve an acceleration operation, a muting operation, and a suppression operation for absorption of electromagnetic energy of an object by adjusting the parameters such as the RF pulse, the gradient field, and the signal acquisition time.

Merely by way of example, during or before a detection process, the first imaging module 220 may determine that the object is unable to cooperate with a long-term scanning due to a physical endurance, a claustrophobia, etc., based on the portrait data of the detection object, and automatically delete non-essential diagnostic scanning protocol(s) in a subsequent scanning phase to shorten the scanning time.

As another example, the first imaging module 220 may change an under-sampling frequency of a scanning protocol in the subsequent scanning phase, and use a machine learning based reconstruction model during a reconstruction process to shorten the scanning time while ensuring diagnostic requirement(s).

In some embodiments of the present disclosure, by determining whether the portrait data of the detection object has changed during the current scanning phase of the initial image scanning scheme, the parameter(s) of at least one pending scanning phase of the image scanning scheme may be optimized. A dynamic adjustment of the image scanning scheme may be achieved based on the changed portrait data of the detection object, which further improves the accuracy of the recommendation of the image scanning scheme and ensures the accuracy of the inspection result.

In some embodiments, the plurality of scanning phases of the initial image scanning scheme may correspond to scanning protocol(s) with the same type. A plurality of scanning protocols with the same type may act on a same region of interest (ROI) of the detection object, and the plurality of scanning protocols in each scanning phase may correspond to the same parameters.

In some embodiments, the first imaging module 220 may adjust the parameters of the plurality of scanning phases corresponding to the scanning protocol(s) with the same type. Taking a magnetic resonance scanning as an example, the first imaging module 220 may perform an initial image scanning scheme to obtain a first set of magnetic resonance signals, and during the current scanning phase, monitor whether a physiological parameter in the portrait data of the detection object has changed. For example, if a heartbeat of the detection object is accelerated, a body temperature of the detection object is elevated, or an emotion of the detection object is irritable, the portrait data of the detection object has changed. In response a determination that the portrait data of the detection object has changed, the first imaging module 220 may adjust a sampling rate of subsequent scanning protocols after the current scanning phase of the image scanning scheme to accelerate scanning. The sampling rate of subsequent scanning protocols may decrease in a stepwise manner, that is, an acceleration factor may increase in a stepwise manner. The first imaging module 220 may execute the adjusted scanning protocol to obtain a second set of magnetic resonance signals and a third set of magnetic resonance signals; reconstruct the first set of magnetic resonance signals, the second set of magnetic resonance signals, and the third set of magnetic resonance signals to obtain the first, second, and third magnetic resonance images; weight and fuse the first, second, and third magnetic resonance images to obtain a target magnetic resonance image of the detection object.

In the embodiments of the present disclosure, the sampling rate may be adjusted in a timely manner based on the physiological state of the detection object, and a target magnetic resonance image with a relatively high signal-to-noise ratio may be obtained within a range of the detection object's bearing capacity.

In some embodiments, the plurality of scanning phases of the initial image scanning scheme may correspond to the plurality of scanning protocols. The plurality of scanning protocols may act on the same ROI of the detection object, forming different contrasts, and the plurality of scanning protocols may have some common parameters. In some embodiments, the first imaging module 220 may adjust the parameters of the plurality of scanning phases corresponding to various types of scanning protocols. Taking a magnetic resonance scanning as an example, assuming that the common parameters among the plurality of scanning phases is a field of view (FOV), the first imaging module 220 may execute the initial image scanning scheme to obtain the first set of magnetic resonance signals, and during the current scanning phase, monitor whether the portrait data of the detection object is moving autonomously. An autonomous motion of the portrait data of the detection object may cause a change in the ROI within the FOV. In some embodiments of the present disclosure, the FOV of the subsequent scanning protocol may be adjusted (e.g., adjusting a direction and/or a center position of FOV) after the current scanning phase of the image scanning scheme in response to a determination that the portrait data of the detection object has changed, so that a center position of the ROI may be always consistent with the center position of the FOV, and the layers of the ROI may match with the direction of the FOV. The adjusted scanning protocol may be executed to obtain the second set of magnetic resonance signals and the third set of magnetic resonance signals. The first set of magnetic resonance signals, the second set of magnetic resonance signals, and the third set of magnetic resonance signals may be used to reconstruct a plurality of magnetic resonance images with multiple contrasts. In some embodiments of the present disclosure, the first imaging module 220 may adjust FOV parameters in a timely manner to match the displaced ROI with the FOV, ensuring consistency between different scanning protocols.

In some embodiments, the first imaging module 220 may store target portrait data and a target image scanning scheme.

The target portrait data may be the portrait data obtained during each scanning phase. In some embodiments, the target portrait data may include, but may not be limited to, a scanning image, scanning data for generating the scanning image, etc. In some embodiments, a format of the scanning image may include the joint photographic experts group (JPEG) format, the tagged image file format (TIFF) format, the graphics interchange format (GIF) format, the kodak flash pix (FPX) format, the digital imaging and communications in medicine (DICOM) format, etc. In some embodiments, the scanning image may be a two-dimensional (2D) image or a three-dimensional (3D) image. In some embodiments, a 3D image may be composed of a series of 2D slices or 2D layers. The target image scanning scheme may be a image scanning scheme obtained by optimizing the parameter(s) of the at least one pending scanning phase. For example, continuing with the above example, the target image scanning scheme may include a scanning protocol with an adjusted FOV.

For the convenience of understanding, the following is an example of a knee joint inspection for an elderly lame detection object.

A first operation is performing a facial recognition on the detection object to obtain facial data of the detection object. Querying is performed in the database based on the facial data of the detection object to obtain twin data of the detection object that matches with the facial data, in which the twin data of the detection object includes: morphological feature data of “elderly, 152 cm, 40-50 kg, female, lame (with pain), walk slowly”; corresponding examination information of the detection object includes “performing a knee joint inspection, being clinically diagnosed as joint degenerative disease”; and the physiological parameters of the detection object include “breathing, heart rate”, etc. Physiological signals relating to the physiological parameters of the detection object may be obtained in real time using a health monitoring equipment after the detection object is lying down on a scanning table of the scanning device.

In a second operation, the above information may be combined with twin data of an operator to form portrait data of the detection object. The portrait data of the detection object may be searched in a historical detection object database to obtain the best practical solution, which may be used as an initial image scanning scheme corresponding to the twin data of the detection object.

In a third operation, the operator may perform the inspection according to the initial image scanning scheme, which may include one or more of the following operations: (1) in a preparation process, considering a situation of an elderly age and a joint pain, strengthening knee joint fixation to avoid involuntary movement during scanning; (2) in system preference setting, playing soothing audio or video in an aperture for the detection object, and automatically increasing a font size; (3) during a current scanning phase, monitoring whether the knee joint of the detection object is moving and a comfort level of the detection object, wherein the comfort level of the detection object may be obtained by monitoring facial expressions of the detection object; (4) in operation navigation, guiding an operation process, prompting whether the scanning region is moving, detecting whether the expression of the object has changed, and automatically providing suggestions. For example, the operation navigation may include providing reminder(s) to the detect object, motion suppression, accelerated acquisition, or the like.

In a fourth operation, during the current scanning phase, if changes are detected in the portrait data of the detection object, one or more parameters of at least one pending scanning phase of the image scanning scheme may be optimized. For example, if it is difficult for the detection object to complete the scanning due to pain and/or discomfort, relevant parameter(s) such as a RF pulse, a gradient field, and/or a signal acquisition time may be adjusted to achieve the adjustment of the initial impact scanning scheme to obtain the target image scanning scheme.

In some embodiments of the present disclosure, by optimizing the portrait data obtained in each scanning phase and the parameter(s) of the at least one pending scanning phase, a image scanning scheme may be obtained and stored, ensuring the reuse of portrait data and image scanning scheme in the future, thus ensuring that the image scanning scheme be dynamically adjusted according to changes in the portrait data of the detection object, improving the accuracy of recommendation of the image scanning scheme.

FIG. 14 is a flowchart illustrating an exemplary process for generating an image scanning scheme based on historical scanning schema data according to some embodiments of the present disclosure. As show in FIG. 14, process 1400 may include one or more of the following operations. In some embodiments, process 1400 may be implemented by a processing device (e.g., the processing device 120) and/or a imaging system 200 (e.g., the second imaging module 224).

In 1410, historical scanning scheme data may be obtained.

The historical scanning scheme data may be data relating to a finished image scanning scheme. In some embodiments, the historical scanning scheme data may include twin data of a historical object and/or twin data of a historical operator.

The twin data of the historical object may be referred to as twin data of a historical detection objecthistorical object. In some embodiments, the twin data of the historical object may include relevant data relating to historical object feature(s) and/or historical inspection site(s).

The historical object feature(s) may be portrait data of of the historical object or a portion of the portrait data of the historical object. The detailed descriptions for the historical object feature may be found elsewhere in the present disclosure, for example, operation 1310 and the relevant descriptions.

In some embodiments, the historical object feature(s) may be divided into or include historical object feature(s) with stable factor(s) and historical object feature(s) with active factor(s). The historical object feature(s) with the stable factor(s) may include body shape data (e.g., a weight, a height, etc.) of the historical object within a preset time period, implant information within a body of the historical object, an age of the historical object, a pregnancy period (a stable feature obtained through calculation) of the historical object, a gender of the historical object, and other feature(s) of the historical object. The historical object feature(s) with the active factor(s) may include pregnancy, fever, pain, unconsciousness, regular breathing, irregular heart rate, and autonomous exercise.

A historical inspection site may refer to an inspection site of the historical object. For example, the heart, the brain, etc.

In some embodiments, the second imaging module 224 may obtain twin data of the historical object from a database (e.g., a hospital's object information database) or other interface(s).

The twin data of the historical operator may refer to operator twin data of the historical operator. The detailed descriptions for the twin data of the historical operator may be found elsewhere in the present disclosure, for example, operation 1320 and the relevant descriptions.

In some embodiments, the second imaging module 224 may obtain the twin data of the historical operator based on a target account. Specifically, the second imaging module 224 may designate an account used by the historical operator as the target account and obtain a target preference setting that matches with the target account from the second device as the twin data of the historical operator. The detailed descriptions for obtaining the target preference setting that matches with the target account may be found elsewhere in the present disclosure, for example, FIGS. 4-8 and the relevant descriptions.

In 1420, portrait data of a current object and initial twin data of a current operator may be obtained.

The portrait data of the current object may refer to portrait data of the current detection object. The detailed descriptions for the portrait data of the current object may be found elsewhere in the present disclosure, for example, operation 1310 and the relevant descriptions.

The initial twin data of the current operator may be or include twin data of a operator preparing to perform a scanning operation. The detailed descriptions for the twin data of the current operator may be found elsewhere in the present disclosure, for example, operation 1320 and the relevant descriptions.

In 1430, the historical scanning scheme data may be fused with the portrait data of the current object and the initial twin data of the current operator to generate an image scanning scheme for the current object.

The image scanning scheme for the current object may be a image scanning scheme to be executed on the current object. In some embodiments, the historical scanning scheme (corresponding to the historical scanning scheme data) and the image scanning scheme for the current object may be executed on different devices. For example, the image scanning scheme for the current object and the historical scanning scheme may be executed on devices of the same type located in different hospitals. For another example, the image scanning scheme for the current object and the historical scanning scheme may be executed on devices located in the same hospital but with different types or modalities.

In some embodiments, the second imaging module 224 may fuse the historical scanning scheme data with the portrait data of the current object and/or the initial twin data of the current operator based on a determination whether the current object and the historical object are the same object, and/or a determination whether a scanning region of interest (ROI) of the current object is the same as a scanning ROI of the historical object, to generate a image scanning scheme for the current object.

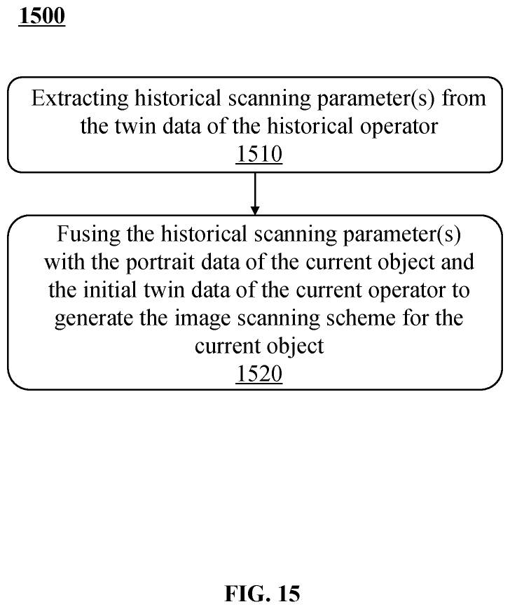

Merely by way of example, the current object and the historical object may be the same object, and the scanning ROI of the current object and the scanning ROI of the historical object may be the same. For example, the current object is the same as the historical object, both being the object a. The scanning ROI of the current object is the same as the scanning ROI of the historical object, both being the heart. The detailed descriptions for the image scanning scheme generated for the current object when the current object and the historical object are the same object, and the scanning ROI of the current object is the same as the scanning ROI of the historical object may be found elsewhere in the present disclosure, for example, FIG. 15 and the relevant descriptions.

As another example, the current object and the historical object may be the same object, and the scanning ROI of the current object and the scanning ROI of the historical object may be different. For example, the current object is the same as the historical object, both being object a. The scanning ROI of the current object is the heart, while the scanning ROI of the historical object is the brain. The detailed descriptions for the image scanning scheme generated for the current object when the current object and the historical object are the same object, and the scanning ROI of the current object is different from the scanning ROI of the historical object may be found elsewhere in the present disclosure, for example, FIG. 15 and the relevant descriptions.

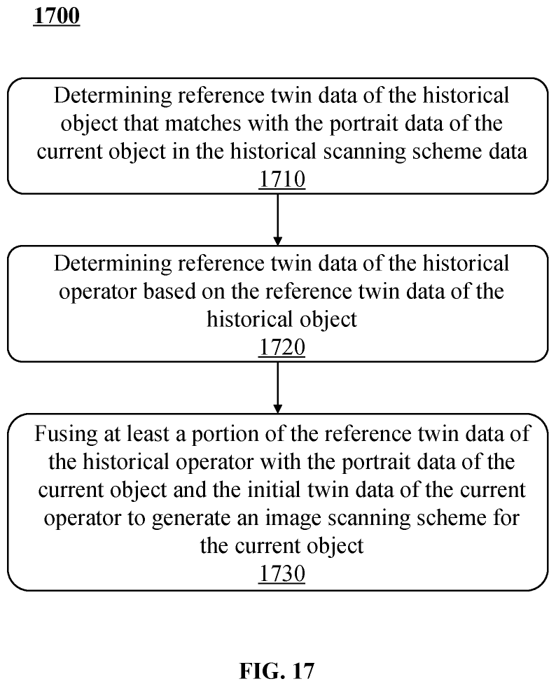

As another example, the current object and the historical object may be different objects, and the scanning ROI of the current object and the Scanning ROI of the historical object may be the same. For example, the current object is the object a, the historical object is an object b, and both the scanning ROI of the current object and the scanning ROI of the historical object are the heart. The detailed descriptions for the image scanning scheme generated for the current object when the current object and the historical object are different objects, and the scanning ROI of the current object is the same as the scanning ROI of the historical object may be found elsewhere in the present disclosure, for example, FIG. 17 and the relevant descriptions.

In some embodiments of the present disclosure, a generation process of the image scanning scheme may include fusing the historical scanning scheme data, the portrait data of the current object and/or the initial twin data of the current operator to generate a image scanning scheme for the current object, thus when an imaging inspection is performed on the current object, a image scanning scheme for the current object may be generated based on the twin data of the historical object, the twin data of the historical operator, the portrait data of the current object and/or the initial twin data of the current operator. There is no need to retest each inspection parameter, thereby improving the efficiency of imaging inspection and reducing the cost of imaging inspection.

In some embodiments, after generating the image scanning scheme for the current object, the second imaging module 224 may use the image scanning scheme for the current object to scan the current object and generate an image scanning result.

In some embodiments, the second imaging module 224 may store the image scanning result. In some embodiments, the image scanning result may include twin data of the current object, imaging data of the current object, and/or twin data of the current operator.

In some embodiments of the present disclosure, the image scanning scheme for the current object may be used to scan the current object and generate the image scanning result for storage, which may ensure that the image scanning scheme of a subsequent object uses relevant data of the image scanning scheme for the current object.

FIG. 15 is a flowchart illustrating an exemplary process for generating an image scanning scheme for a current object when the current object is the same as a historical object, and a scanning region of interest (ROI) of the current object is the same as a scanning ROI of the historical object. As show in FIG. 15, process 1500 may include one or more of the following operations. In some embodiments, process 1500 may be implemented by a processing device (e.g., the processing device 120) and/or a imaging system 200 (e.g., the second imaging module 224).

In 1510, historical scanning parameter(s) may be extracted from twin data of a historical operator.

The historical scanning parameter(s) may be or include operational parameter(s) the historical operator used in executing a historical scanning scheme. In some embodiments, the second imaging module 224 may extract the historical scanning parameter(s) based on data requirements of a current operator. For example, if the current operator A is uncertain about the scanning parameter “pulse sequence type”, the second imaging module 224 may extract a numerical value corresponding to the historical scanning parameter “pulse sequence type” from the twin data of a plurality of historical operators. The historical operator may be the same as or different from the current operator.

In 1520, the historical scanning parameter(s) may be fused with portrait data of a current object and/or initial twin data of the current operator to generate a image scanning scheme for the current object.

Combined with the descriptions in FIG. 3, the initial twin data of the current operator may be or include twin data of an operator migrated from a second device based on a target account by the current operator. In some embodiments, the second imaging module 224 may replace one or more corresponding scanning parameters in the initial twin data of the current operator with the historical scanning parameter(s). Continuing with the above example, if the “pulse sequence type” in the initial twin data of the current operator is EPI, and the historical scanning parameter “pulse sequence type” is mostly GRE, then the second imaging module 224 may replace EPI with GRE.

In some embodiments, the second imaging module 224 may adjust one or more scanning parameters in the initial twin data based on the portrait data of the current object. The detailed descriptions for fusing the portrait data of the current object and the initial twin data of the current operator may be found elsewhere in the present disclosure, for example, operation 1320 and the relevant descriptions.

In some embodiments, the second imaging module 224 may initiate a confirmation request to a user before fusing the historical scanning parameter(s) with the portrait data of the current object and/or the initial twin data of the current operator. After receiving confirmation information from the user, the fusing operation may be performed.

In some embodiments of the present disclosure, by extracting the historical scanning parameter(s) from the twin data of the historical operator and fusing them with the portrait data of the current object and/or the initial twin data of the current operator, the image scanning scheme for the current object may generated, thereby achieving an effect that when the current object and the historical object are the same object, and the scanning ROI of the current object is the same as the scanning ROI of the historical object, the twin data of the historical operator may be reused to generate the image scanning scheme for the current object, thus improving the efficiency of imaging inspection and reducing the cost of imaging inspection.

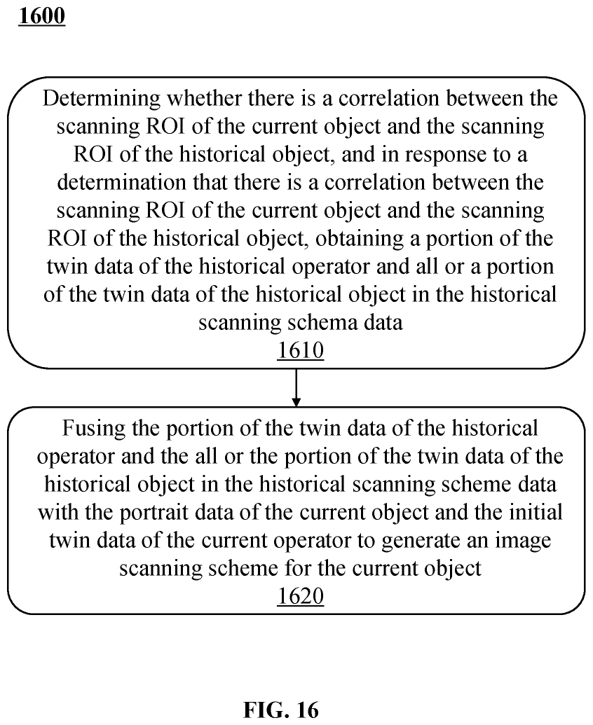

FIG. 16 is a flowchart illustrating an exemplary process for generating an image scanning scheme for a current object when the current object is the same as a historical object, and a scanning ROI of the current object is different from a scanning ROI of the historical object. As show in FIG. 16, process 1600 may include one or more of the following operations. In some embodiments, process 1600 may be implemented by a processing device (e.g., the processing device 120) and/or a imaging system 200 (e.g., the second imaging module 224).

In 1610, whether there is a correlation between the scanning ROI (e.g., a scanning site) of the current object and the scanning ROI (e.g., a scanning site) of the historical object may be determined. In response to a determination that there is a correlation between the scanning ROI of the current object and the scanning ROI of the historical object, a portion of the twin data of the historical operator and all or a portion of the twin data of the historical object in the historical scanning schema data may be obtained.

The correlation between the scanning ROI of the current object and the Scanning ROI of the historical object may be that the scanning ROI of the current object and the scanning ROI of the historical object are the same part of a body. For example, a part of the body of the scanning ROI may include a relatively large part such as the head, chest, abdomen, limbs, etc. For example, if the scanning ROI of the current object is the brainstem, and the scanning ROI of the historical objects is the cerebellum, then the scanning ROI of the current object and the scanning ROI of the historical object may be the same body part.

In some embodiments, the second imaging module 224 may determine whether there is a correlation between the scanning ROI of the current object and the scanning ROI of the historical object by the following operations:

In a first operation, identity information, a device type, an inspection site, and a disease category of the current object may be obtained.

In a second operation, fields of the identity information, the device type, and the inspection site may be extracted.

In a third operation: a historical object that is the same as the current object may be queried based on the identity information of the current object, and the fields of the identity information of the historical object, the device type, and the inspection site may be extracted.

In a fourth operation: whether the fields of the identity information, the device type, and the inspection site of the current object are the same as the fields of the identity information of historical object, the device type, and the inspection site of the historical object may be determined. In response to a determination that the fields of the identity information, the device type, and the inspection site of the current object are the same as the fields of the identity information of historical object, the device type, and the inspection site of the historical object, whether there is a correlation between the scanning ROI of the current object and the scanning ROI of the historical object may be determined.

The portion of twin data of the historical operator may be specific data from the twin data of the historical operator. In some embodiments, the portion of the twin data of the historical operator may include at least one of a positioning way, a scanning navigation parameter, a RF parameter threshold, and a gradient parameter threshold, etc. In some embodiments, the second imaging module 224 may obtain the portion of the twin data of the historical operator from a database (e.g., an operator operation backend database) that stores twin data of a plurality of historical operators.

All twin data of the historical object(s) may include all twin data of a plurality of historical objects. The detailed descriptions for obtaining all twin data of the historical object may be found elsewhere in the present disclosure, for example, operation 1410 and the relevant descriptions.

The portion of twin data of the historical object may be specific data from twin data of a plurality of historical objects. In some embodiments, the second imaging module 224 may obtain the portion of twin data of the historical object based on the twin data of the historical object.

Specifically, in some embodiments, the second imaging module 224 may obtain historical portrait data of the object from the twin data of the historical object, and then analyze a change of portrait data of the current object in a current inspection compared with the historical portrait data to obtain an analysis result. Merely by way of example, the current object may be the same as the historical object, both being the object a. The second imaging module 224 may obtain the historical portrait data (e.g., there is no implant in the body of the object a) of the object a from the twin data of object a, and then analyze the portrait data of object a in the current inspection (e.g., there is a metal implant in the body of the object a) to obtain the analysis result of “new metal implant in the body of the object a”.

In some embodiments, the second imaging module 224 may send the analysis result to a terminal for display, enabling an operator to visually see the analysis result, facilitating operator's confirmation, and further ensuring the accuracy of obtaining the portion of the twin data of the historical object.

In some embodiments, the second imaging module 224 may obtain, based on the analysis result, twin data of the historical objects with a stability factor from the twin data of the historical object as the portion of the twin data of the historical object. Combined with description in operation 1410, the twin data of the historical objects with a stability factor in the twin data of the historical object may include at least one of body shape data of the historical object, implant information of the historical object, and an age of the historical object within a preset time period, etc. For example, the twin data of the historical objects with a stability factor may include: weight and height information of the object a within one month, and metal implant information in object a. The second imaging module 224 may obtain the portion of the twin data of object a (e.g., the weight and height information of the object a) based on the analysis result of “new metal implant in the body of the object a”.

In some embodiments of the present disclosure, by comparing the portrait data of the current object with the historical portrait data, the twin data of the historical objects with a stability factor may be obtained as the portion of the twin data of the historical object, thereby ensuring the accuracy of obtaining the portion of the twin data of the historical object and ensuring the accuracy of subsequent generation of a image scanning scheme for the current object.

In 1620, the portion of the twin data of the historical operator and the all or the portion of the twin data of the historical object in the historical scanning scheme data may be fused with the portrait data of the current object and the initial twin data of the current operator to generate the image scanning scheme for the current object.

Specifically, the second imaging module 224 may replace the initial twin data of the current operator with the portion of the twin data of the historical operator, and combine the portion of the twin data of the historical operator with the portrait data of the current object to generate an initial image scanning scheme. The detailed descriptions for replacing the initial twin data of the current operator with the image twin data of the historical operator may be found elsewhere in the present disclosure, for example, operation 1520 and the relevant descriptions.

Furthermore, the second imaging module 224 may adjust the initial image scanning scheme based on a change in all or the portion of the twin data of the historical object and the portrait data of the current object, and generate the image scanning scheme for the current object. For example, based on the analysis result of “new metal implant in the body of the object a”, an acquisition scheme with metal artifact suppression may be automatically selected.

In some embodiments of the present disclosure, by obtaining the portion of twin data of the historical operator and all or the portion of the twin data of the historical object from the historical scanning scheme data, and fusing the portion of the twin data of the historical operator and the all or the portion of the twin data of the historical object in the historical scanning scheme data with the portrait data of the current object and the initial twin data of the current operator to generate a image scanning scheme for the current object, thus when the current object and the historical object are the same object, and the scanning ROI of the current object is different from the scanning ROI of the historical object, and there is a correlation between the scanning ROI of the object and the scanning ROI of the historical object, the twin data of historical operator and the twin data of historical object may be reused to generate the image scanning scheme for the current object, thus improving the efficiency of imaging inspection and reduces the cost of imaging inspection.

In some embodiments, if the portrait data of the current object is the same as the historical portrait data, but a lesion area has changed due to radiotherapy or chemotherapy, or the lesion area has metastasized, the following operations may be performed: after obtaining historical data for reuse, if the lesion area has changed or metastasized to another area during a scanning process, an operator may adjust a scanning scheme based on a specific situation. For example, when the lesion area has metastasized, a local scanning for an area of the new lesion may be added. Alternatively, if the lesion area has metastasized to a plurality of areas, a scan for full body may be performed.

In some embodiments, if the scanning site of the current object is different from the scanning site of the historical object, there may be no correlation between the scanning ROI of the object and the scanning ROI of the historical object, the second imaging module 224 may obtain the portion of twin data of the historical operator and the portion of the twin data of the historical object from the historical scanning scheme data, and fuse the portion of the twin data of the historical operator and the portion of the twin data of the historical object with the portrait data of the current object and the initial twin data of the current operator to generate the image scanning scheme for the current object.

In some embodiments of the present disclosure, by obtaining all twin data of the historical operator and all twin data of the historical object from the historical scanning scheme data, and fusing all twin data of the historical operator and all twin data of the historical object with the portrait data of the current object and the initial twin data of the current operator, the image scanning scheme for the current object may be generated. When the current object and the historical object are the same object, and the scanning ROI of the current object is different from the scanning ROI of the historical object, and there is a correlation between the scanning ROI of the current object and the scanning ROI of the historical object, it is possible to partially reuse the twin data of the historical operator and the twin data of the historical object to generate the image scanning scheme for the current object, thereby improving the efficiency of imaging inspection and reducing the cost of imaging inspection.

FIG. 17 is a flowchart illustrating an exemplary process for generating an image scanning scheme for a current object when the current object is different from a historical object, and a scanning ROI of the current object is the same as a scanning ROI of the historical object.

In some embodiments, the second imaging module 224 may determine whether the scanning ROI of the current object is the same as the scanning ROI of the historical object based on similar cases of different objects. Specifically, the determination operations may include: obtaining a corresponding device type, an inspection site, and a disease category of the current object; extracting fields of the device type, the inspection site, and the disease category, respectively; querying the historical scan scheme data for matched scanning scheme data based on the fields of the device type, the inspection site, and the disease category; if the matched scanning scheme data is found in the historical scanning scheme data based on the fields of the device type, the inspection site, and the disease category (the device types, the inspection sites, and the disease categories of the current object and the historical object are all consistent), it may be determined that there is a correlation between the scanning ROI of the current object and the scanning ROI of the historical object. That is, the current object may be different from the historical object, but the scanning ROI of the current object is the same as the scanning ROI of the historical object.

As shown in FIG. 17, process 1700 may include one or more of the following operations. In some embodiments, process 1700 may be implemented by a processing device (e.g., the processing device 120) and/or a imaging system 200 (e.g., the second imaging module 224).

In 1710, reference twin data of the historical object that matches with the portrait data of the current object may be determined in the historical scanning scheme data.

The reference twin data of the historical object may be twin data of an object different from the current object. In some embodiments, the second imaging module 224 may retrieve the twin data of the historical object with a highest similarity to the portrait data of the current object from the historical scanning scheme data as the reference twin data of the historical object. For example, in the historical scanning scheme data, the twin data of an object b with the highest similarity to the current portrait data of the current object a may be used as the reference twin data of the historical object.

In 1720, reference twin data of the historical operator may be determined based on the reference twin data of the historical object.

The reference twin data of the historical operator may be a preference setting of the historical operator during an execution of a historical image scanning scheme that differ from a preference setting of the current operator in a current scanning scheme.

In some embodiments, the second imaging module 224 may obtain the historical image scanning scheme based on the reference twin data of historical object, and then obtain the reference twin data of historical operator from the historical image scanning scheme.

For example, continuing with the above example, the reference twin data of the historical object may include the twin data of the object b. The second imaging module 224 may obtain the historical image scanning scheme of the object b from the historical twin data of the object b, then obtain, from the historical image scanning scheme of the object b, a preference setting of the historical operator (which may be the same as or different from the current operator) that is different from the current preference setting of the current operator, and designate the preference setting of the historical operator as the reference twin data of the historical operator.

In 1730, at least a portion of the reference twin data of the historical operator may be fused with the portrait data of the current object and/or the initial twin data of the current operator to generate a image scanning scheme for the current object.

In some embodiments, the second imaging module may replace the at least a portion of the reference twin data of the historical operator with the initial twin data of the current operator, and then adjust parameter(s) of the initial image scanning scheme based on the portrait data of the current object to obtain the image scanning scheme for the current object.

In some embodiments of the present disclosure, by determining the reference twin data of the historical object that matches with the portrait data of the current object in the historical scanning scheme data, and then determining the reference twin data of the historical operator based on the reference twin data of the historical object, and fusing the at least a portion of the reference twin data of the historical operator with the portrait data of the current object and/or the initial twin data of the current operator to generate the image scanning scheme for the current object, the goal of reusing the twin data of historical operator and historical object to generate image scanning scheme for the current object can be achieved when the current object is different from the historical object, and the scanning ROI of the current object is the same as the scanning ROI of the historical object, thus improving the imaging efficiency and reducing the imaging costs.

The beneficial effects the embodiments of the present disclosure may include, but may not be limited to: (1) the first device can obtain the target preference setting via an interaction with the server; specifically, for a second device, the user may start the second device using the user's account and complete the setting of configuration option(s) on the second device; the second device may send the configuration data to the server, enabling the server to establish the first mapping relationship based on the received configuration data; after the server receives the request sent by the first device, the target preference setting may be searched from the first mapping relationship, and then returned to the first device, allowing the user to migrate a configuration made on the second device to the first device without the need for the user to make other configuration(s) on the first device, saving a lot of workload and improving the user experience; (2) the generation process of the image scanning scheme may include fusing the historical scanning scheme data, the portrait data of the current object and/or the initial twin data of the current operator to generate a image scanning scheme for the current object, thus when an imaging inspection is performed on the current object, a image scanning scheme for the current object may be generated based on the twin data of the historical object, the twin data of the historical operator, the portrait data of the current object, and/or the initial twin data of the current operator; there is no need to retest each inspection parameter, thereby improving the efficiency of imaging inspection and reducing the cost of imaging inspection.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. Furthermore, the particular features, structures or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method implemented on at least one machine each of which has at least one processor and at least one storage device for imaging, comprising:
obtaining historical configuration information, the historical configuration information including at least one of a historical inspection process configuration, a historical scanning scheme configuration, a historical displaying configuration, or twin data of a historical detection object used when a historical scanning device is running in a historical scanning condition;
adjusting the historical configuration information to generate target configuration information matching a current scanning condition, the target configuration information including at least one of an inspection process configuration, a scanning scheme configuration, a displaying configuration, or twin data of a detection object; and
performing, using a first device, an imaging operation on the detection object based on the target configuration information, wherein
an account, a current scanning device, an operator, or a detection object under the current scanning condition is different from an account, the historical scanning device, an operator, or a detection object under the historical scanning condition, respectively.

2. (canceled)

3. The method of claim 1, wherein the performing, using a first device, an imaging operation on the detection object based on the target configuration information includes:
updating a setting corresponding to at least one configuration option on the first device based on a target preference setting related to the inspection process configuration, the scanning scheme configuration, and/or the displaying configuration in the target configuration information.

4. The method of claim 3, wherein the target preference setting includes a setting of one or more configuration options on a second device, the setting of the one or more configuration options being set by an operator on the second device using a target account.

5. (canceled)

6. The method of claim 1, further comprising:
determining, based on the twin data of the detection object, in the scanning scheme configuration, an initial image scanning scheme corresponding to the twin data of the detection object, the initial image scanning scheme including a plurality of scanning phases.

7. The method of claim 6, wherein the twin data of the detection object includes portrait data of the detection object, and the performing, using a first device, an imaging operation on the detection object based on the target configuration information includes:
executing the initial image scanning scheme;
determining whether the portrait data of the detection object has changed during a current scanning phase of the plurality of scanning phases; and
in response to a determination that the portrait data of the detection object has changed, optimizing one or more parameters of at least one pending scanning phase of the plurality of scanning phases in the initial image scanning scheme.

8. The method of claim 1, further comprising:
obtaining historical scanning scheme data, the historical scanning scheme data including twin data of a historical object and/or twin data of a historical operator.

9. (canceled)

10. (canceled)

11. (canceled)

12. A system for imaging, comprising:
an acquisition module configured to obtain historical configuration information, and adjust the historical configuration information to generate target configuration information matching a current scanning condition; and
an imaging module configured to perform an imaging operation using a first device based on the target configuration information, wherein the target configuration information includes at least one of inspection process configuration, scanning scheme configuration, displaying configuration, and twin data of a detection object.

13. A non-transitory computer readable medium storing instructions, the instructions, when executed by at least one processor, causing the at least one processor to implement a method including:
obtaining historical configuration information, the historical configuration information including at least one of a historical inspection process configuration, a historical scanning scheme configuration, a historical displaying configuration, or twin data of a historical detection object used when a historical scanning device is running in a historical scanning condition;
adjusting the historical configuration information to generate target configuration information matching a current scanning condition, the target configuration information including at least one of an inspection process configuration, a scanning scheme configuration, a displaying configuration, or twin data of a detection object; and
performing, using a first device, an imaging operation on the detection object based on the target configuration information, wherein
an account, a current scanning device, an operator, or a detection object under the current scanning condition is different from an account, the historical scanning device, an operator, or a detection object under the historical scanning condition, respectively.

14. The method of claim 1, further comprising a method implemented on a first device for data migration, comprising:
obtaining a target account via a login interface of the first device;
obtaining a target preference setting corresponding to the target account, the target preference setting including a setting of one or more configuration options on a second device, the setting of the one or more configuration options being set by an operator on the second device after the operator starts the second device using the target account, the one or more configuration options including at least one of an inspection process configuration, a scanning scheme configuration, or a displaying configuration;
updating a setting corresponding to at least one configuration option on the first device based on the target preference setting.

15. The method of claim 14, wherein the obtaining a target preference setting corresponding to the target account includes:
sending a first acquisition request to a server, wherein the first acquisition request carries the target account to facilitate the server to search, based on the target account and a first mapping relationship, for the target preference setting corresponding to the target account, the first mapping relationship indicating a correlation between a plurality of accounts and a plurality of preference settings; and
receiving the target preference setting sent by the server.

16. The method of claim 14, further comprising:
obtaining a device identification of the second device;
determining, based on the device identification of the second device, whether the target preference setting is compatible with the first device; and
in response to a determination that the target preference setting is incompatible with the first device,
adjusting the target preference setting; and
updating the setting corresponding to the at least one configuration option on the first device based on the adjusted target preference setting.

17. (canceled)

18. The method of claim 1, further comprising a A-method implemented on a server for data migration, comprising:
receiving a first acquisition request sent by a first device, wherein the first acquisition request carries a target account, the target account being obtained via a login interface of the first device;
searching, based on the target account and a first mapping relationship, for a preference setting corresponding to the target account, the first mapping relationship indicating a correlation between a plurality of accounts and a plurality of preference settings;
designating the preference setting as a target preference setting corresponding to the target account, wherein the target preference setting includes a setting of one or more configuration options on a second device, the setting of the one or more configuration options being set by an operator on the second device using the target account, and the one or more configuration options including at least one of an inspection process configuration, a scanning scheme configuration, or a displaying configuration;
sending the target preference setting to the first device to facilitate the first device to update a setting corresponding to at least one configuration option on the first device based on the target preference setting; and
starting the first device.

19. The method of claim 18, further comprising:
obtaining a first device identification of the first device and a second device identification of the second device, respectively;
determining, based on the first device identification and the second device identification, whether the target preference setting is compatible with the first device; and
in response to a determination that the target preference setting is incompatible with the first device,
adjusting the target preference setting; and
updating the setting corresponding to the at least one configuration option on the first device based on the adjusted target preference setting.

20. The method of claim 1, further comprising a A-method implemented on a second device for data migration, comprising:
receiving a second acquisition request sent by a first device, wherein the second acquisition request carries a target account, and the second acquisition request is sent by the first device based on a device identification of the second device;
searching, based on the target account and a second mapping relationship, for a preference setting corresponding to the target account, the second mapping relationship indicating correlations between a plurality of accounts and a plurality of preference settings;
designating the preference setting as a target preference setting corresponding to the target account, wherein the target preference setting includes a setting of one or more configuration options on a second device, the setting of the one or more configuration options being set by an operator on the second device using the target account, and the one or more configuration options including at least one of an inspection process configuration, a scanning scheme configuration, or a displaying configuration;
sending the target preference setting to the first device to facilitate the first device to update a setting corresponding to at least one configuration option on the first device based on the target preference setting; and
starting the first device.

21. (canceled)

22. (canceled)

23. The method of claim 1, further comprising a terminal, comprising:
a receiving module configured to receive a second configuration acquisition request sent by a first device, wherein the second configuration acquisition request carries a target account, and the second configuration acquisition request is sent by the first device based on a device identification of a second device;
a searching module configured to search for a preference setting corresponding to a target account in a second mapping relationship based on the target account, and designate the preference setting as the target preference settings, wherein the second mapping relationship is used to indicate a correlation between an account and a preference setting, and the target preference setting is a result of setting a configuration option on a second device after a user starts the second device using the target account, and the configuration option include at least one of inspection process configuration, scanning scheme configuration, and displaying configuration;
a sending module configured to send the target preference setting to the first device to drive the first device to update a setting corresponding to a configuration option on the first device based on the target preference setting, and start the first device.

24. The method of claim 1, further comprising:
obtaining twin data of a detection object, the twin data of the detection object including portrait data of the detection object;
determining, based on the twin data of the detection object, an initial image scanning scheme corresponding to the twin data of the detection object, the initial image scanning scheme including a plurality of scanning phases;
executing the initial image scanning scheme;
determining whether the portrait data of the detection object has changed during a current scanning phase of the plurality of scanning phases;
in response to a determination that the portrait data of the detection object has changed, optimizing one or more parameters of at least one pending scanning phase of the plurality of scanning phases in the initial image scanning scheme.

25-33. (canceled)

34. The method of claim 1, further comprising:
obtaining historical scanning scheme data, the historical scanning scheme data including twin data of a historical object and twin data of a historical operator;
obtaining portrait data of a current object and initial twin data of a current operator;
fusing the historical scanning scheme data with the portrait data of the current object and the initial twin data of the current operator to generate an image scanning scheme for the current object.

35. The method of claim 34, wherein in response to the current object being the same as the historical object, and a scanning region of interest (ROI) of the current object is the same as a scanning ROI of the historical object, the fusing the historical scanning scheme data with the portrait data of the current object and the initial twin data of the current operator includes:
extracting one or more historical scanning parameters from the twin data of the historical operator; and
fusing the one or more historical scanning parameters with the portrait data of the current object and the initial twin data of the current operator to generate the image scanning scheme for the current object.

36. The method of claim 34, wherein in response to the current object being the same as the historical object, and a scanning region of interest (ROI) of the current object is different from a scanning ROI of the historical object, the fusing the historical scanning scheme data with the portrait data of the current object and the initial twin data of the current operator includes:
determining whether there is a correlation between the scanning ROI of the current object and the scanning ROI of the historical object;
in response to a determination that there is a correlation between the scanning ROI of the current object and the scanning ROI of the historical object, obtaining a portion of the twin data of the historical operator and all or a portion of the twin data of the historical object in the historical scanning scheme data; and
fusing the portion of the twin data of the historical operator and the all or the portion of the twin data of the historical object in the historical scanning scheme data with the portrait data of the current object and the initial twin data of the current operator to generate the image scanning scheme for the current object.

37. (canceled)

38. (canceled)

39. The method of claim 34, wherein in response to the current object being different from the historical object, and a scanning region of interest (ROI) of the current object is the same as a scanning ROI of the historical object, the fusing the historical scanning scheme data with the portrait data of the current object and the initial twin data of the current operator includes:
determining reference twin data of the historical object that matches with the portrait data of the current object in the historical scanning scheme data;
determining reference twin data of the historical operator based on the reference twin data of the historical object; and
fusing at least a portion of the reference twin data of the historical operator with the portrait data of the current object and the initial twin data of the current operator to generate the image scanning scheme for the current object.

40-43. (canceled)
